(19) United States
(12) Patent Application Publication
(10) Pub. No.: US 2021/0324426 A1
(43) Pub. Date: Oct. 21, 2021

(54) WHOLE-CELL BIOCATALYSIS METHOD FOR PRODUCING ALPHA, OMEGA-DICARBOXYLIC ACIDS AND USE THEREOF

(71) Applicant: Hubei University, Wuhan City (CN)

(72) Inventors: Aitao Li, Wuhan City (CN); Jing Zhao, Wuhan City (CN); Fei Wang, Wuhan City (CN); Qian Li, Wuhan City (CN); Xiaojuan Yu, Wuhan City (CN); Xueying Peng, Wuhan City (CN); Xiaoman Chen, Wuhan City (CN)

(73) Assignee: Hubei University, Wuhan City (CN)

(21) Appl. No.: 17/158,893

(22) Filed: Jan. 26, 2021

(30) Foreign Application Priority Data
Mar. 3, 2020 (CN) 202010139295.6

(51) Int. Cl. C12P 7/44; C12P 17/08; C12P 7/02; C12N 9/06; C12N 9/04; C12N 9/02; C12N 9/18

(52) U.S. Cl. CPC C12P 7/44; C12P 17/08; C12P 7/02; C12N 9/0016; C12Y 104/01003; C12Y 301/01025; C12Y 101/01001; C12N 9/0071; C12Y 114/14; C12N 9/18; C12N 9/0006

(57) ABSTRACT

The present disclosure belongs to the technical field of biocatalysis and biotransformation, and particularly relates to whole-cell biocatalysis method for producing α, ω-dicarboxylic acids and use thereof. The biosynthetic pathway designed in the present disclosure is divided into three modules to co-express several different enzymes in host cells respectively, and then the whole-cells are used to catalyze the production of α, ω-dicarboxylic acid from cycloalkanes, cycloalkanol and lactones in a cascade reaction. Compared with the chemical method, this process does not produce any harmful gases during the production process, does not require high temperature, high pressure, and complex metal catalysts, and is a green and environmental protection production method.

### CROSS REFERENCE TO RELATED APPLICATION

This disclosure claims the priority of Chinese Patent Application NO. 202010139295.6 entitled “Whole-cell biocatalysis method for producing α, ω-dicarboxylic acids and use thereof” filed with China National Intellectual Property Administration on Mar. 3, 2020, which is incorporated herein by reference in its entirety.

### TECHNICAL FIELD

The present disclosure belongs to the technical field of biocatalysis and biotransformation, and particularly relates to a whole-cell biocatalysis method for producing α, ω-dicarboxylic acids and use thereof.

### BACKGROUND ART

Aliphatic α, ω-dicarboxylic acids (DCAs) are an important class of platform chemical products, which are widely used in perfumes, polymers, adhesives, and macrolide antibiotics. Among those chemicals, adipic acid (or adipate) has received much attention. Adipic acid, also known as fatty acid, is an important dicarboxylic acid, which has the ability to undergo salt formation, esterification, and amidation reaction, etc., and can be polycondensed into high polymer with diamine or diol. At present, the most important application field of adipic acid is synthetic nylon fibers (such as nylon 6,6). It is estimated that the global market value of adipic acid will reach 6.3 billion U.S. dollars by 2018, and the output will be approximately 2.56 billion kilograms, which is still increasing, and the new production capacity in the past two years is mainly in China. Other dicarboxylic acids are also important chemical raw materials and are widely used in chemistry, medicine, construction, and agriculture, etc. For example, glutaric acid can be used as an intermediate of plasticizers in the plastics industry, and has a broad-spectrum bactericidal function in medicine field. Pimelic acid is widely used in industry to synthesize lubricating oil, plasticizer, heat transfer oil, dielectric fluid, surfactant, resin, etc. Suberic acid can also be used in the plastics industry to prepare alkyd resins, fuels, etc.

At present, adipic acid, which is the most concerned among these α, ω-dicarboxylic acids, is mainly synthesized by chemical methods using petrochemical materials (such as nitric acid oxidation process using KA oil, a mixture of cyclohexanol and cyclohexanone, as the raw material). However, the chemical method has problems such as lengthy production process, harsh reaction conditions, many by-products, serious emission of “three wastes”, and the emission of a large amount of greenhouse gases. Therefore, in recent years, more and more people have focused on the production of adipic acid by biotransformation using different substrates and different strains. These processes often require complex engineering design of microorganisms through metabolic engineering and synthetic biology techniques. At the same time, there are still some problems such as the constraints of redox reaction, the identification and engineering of enzymes in metabolic pathways, the selection of suitable host bacteria and metabolic pathways, as well as the potential problems of cell culture and difficulty in purification of fermentation broth at a later stage.

Therefore, a green and efficient whole-cell biocatalysis method for producing α, ω-dicarboxylic acid is urgently needed in the art to promote the industrial production of dicarboxylic acids.

### SUMMARY OF THE INVENTION

The purpose of the disclosure is to provide a whole-cell biocatalysis method for producing α, ω-dicarboxylic acids and use thereof, so as to solve/alleviate some of the problems in the prior art.

The present disclosure is realized in this way that a whole-cell biocatalysis method for producing α, ω-dicarboxylic acids, comprises the following steps of under normal temperature and pressure and aerobic conditions, catalytically converting substrate lactones to obtain α, ω-dicarboxylic acids by recombinant cells containing functional genes related to the pathway for catalyzing lactones to produce α, ω-dicarboxylic acids.

In some embodiments, when cycloalkanols are used as substrates, the disclosure also includes recombinant cells containing functional genes related to the pathway for catalyzing cycloalkanol to produce lactones.

The functional genes related to the pathway for catalyzing cycloalkanol to produce lactones and the functional genes related to the pathway for catalyzing lactones to produce α, ω-dicarboxylic acids are located in the same cell, and the catalysis and transformation of substrates are realized by using a single cell system.

Or the functional genes related to the pathway for catalyzing cycloalkanol to produce lactones and the functional genes related to the pathway for catalyzing lactones to produce α, ω-dicarboxylic acids are respectively constructed in different cells, and the catalysis and transformation of substrates are realized by using a multi-cell combination system.

In some embodiments, when cycloalkanes are used as substrates, the disclosure also includes recombinant cells containing functional genes related to the pathway for catalyzing cycloalkane to produce cycloalkanol.

The functional genes related to the pathway for catalyzing cycloalkane to produce cycloalkanol, the functional genes related to the pathway for catalyzing cycloalkanol to produce lactones and the functional genes related to the pathway for catalyzing lactones to produce α, ω-dicarboxylic acids are located in the same cell, and the catalysis and transformation of substrates are realized by using a single cell system.

Or the functional genes related to the pathway for catalyzing cycloalkane to produce cycloalkanol, the functional genes related to the pathway for catalyzing cycloalkanol to produce lactones and the functional genes related to the pathway for catalyzing lactones to produce α, ω-dicarboxylic acids are respectively located in different cells, and the catalysis and transformation of substrates are realized by using a multi-cell combination system.

Or the functional genes related to the pathway for catalyzing cycloalkane to produce cycloalkanol, the functional genes related to the pathway for catalyzing cycloalkanol to produce lactones and the functional genes related to the pathway for catalyzing lactones to produce α, ω-dicarboxylic acids, any two of which are located in the same cell, and the other one is located in another cell, the catalysis and transformation of substrates are realized by using a multi-cell combination system.

In some embodiments, the α, ω-dicarboxylic acids include different dicarboxylic acids of the C5, C6, C7, C8, C10, C12 and C15 classes.

In some embodiments, the functional genes related to the pathway for catalyzing lactones to produce α, ω-dicarboxylic acids include lactonase gene, alcohol dehydrogenase gene, aldehyde dehydrogenase gene and NADH oxidase gene. The lactonase gene is lactonase, Rhodococcus sp. HI-31, the alcohol dehydrogenase gene is ADH2, Acinetobacter sp. NCIMB9871, the aldehyde dehydrogenase gene is ALDH, Acinetobacter sp. NCIMB9871, the NADH oxidase gene is NOX and Lactobacillus brevis DSM 20054.

In some embodiments, the functional genes related to the pathway for catalyzing cycloalkanol to produce lactones include alcohol dehydrogenase gene and Baeyer-Villiger monooxygenase gene. The alcohol dehydrogenase is ADH1 and Lactobacillus brevis ATCC 14869, the Baeyer-Villiger monooxygenase gene is BVMO, Acinetobacter sp. NCIMB9871, and contains double mutation site C376I/M400I.

In some embodiments, the functional genes related to the pathway for catalyzing cycloalkane to produce cycloalkanol comprise any one of P450_{BM3}19A12 gene, P450_{BM3} A82F gene and P450_{BM3} A82F/A328F gene, further comprise glucose dehydrogenase gene GDH.

In some embodiments, adding glucose solution into the catalytic reaction solution when the substrates are cycloalkane.

In some embodiments, culturing the cells containing functional genes related to the pathway for catalyzing corresponding substrates to produce α, ω-dicarboxylic acids in TB liquid medium, and adding an IPTG or lactose inducer to induce expression; collecting the cultured cells for substrate conversion.

In some preferred embodiments, the cell is any one from Escherichia coli (E. coli), Corynebacterium glutamicum, Bacillus subtilis, Brevibacterium flavum, Serratia marcescens, and Saccharomyces cerevisiae; in a more preferred embodiment, the cell is Escherichia coli (E. coli) cell.

In some embodiments, the catalytic reaction is carried out under normal temperature, normal pressure and aerobic conditions, and in some specific embodiments, the catalytic reaction is carried out in a temperature range of 20° C.-40° C.; preferably in a temperature range of 25° C.-30° C.; and more preferably at 25° C.

A use of the whole-cell biocatalysis method for producing α, ω-dicarboxylic acids as described above in the production of α, ω-dicarboxylic acids.

A use of the whole-cell biocatalysis method for producing α, ω-dicarboxylic acids as described above in the production of any one of C5, C6, C7 or C8 diacid products.

A use of recombinant cells in the whole-cell biocatalysis method for producing α, ω-dicarboxylic acids as described above in the production of immobilized cells.

The present disclosure takes the production of adipic acid as a characteristic reaction and uses a whole-cell one-pot method to produce adipic acid with cyclohexane, cyclohexanol and F-caprolactone as initial substrate respectively. The cells that react with cyclohexanol (as the substrate) are named E. coli (M2E_M3J), which contain module II and module III. 3 mM of adipic acid can be obtained from 50 mM cyclohexanol without any optimization, which proves that the conversion of cyclohexanol to adipic acid can be realized by using single cell. The cells that react with cyclohexane (as the substrate) are named E. coli (M12A_M3J), which contain module I, module II and module III. 4 mM of adipic acid can be obtained from 50 mM cyclohexane without any optimization, which proves that the conversion of cyclohexane to adipic acid can be realized by using single cell. The cells that react with caprolactone (as the substrate) only contains module III, and adipic acid with high concentration can be obtained without any optimization. Therefore, we optimize the process using caprolactone as the substrate, and the conversion of final adipic acid could reach 63 g/L by batch feeding.

In summary, the advantages and positive effects of the present disclosure are:

A: Compared with Traditional Chemical Methods in Industry:

1. The whole reaction of the present disclosure is carried out under normal temperature and pressure, to avoid energy consumption problems caused by high temperature and high pressure, and do not involve harsh reaction conditions.

2. No harmful raw materials, such as nitric acid required by chemical methods and the like, are used in the whole reaction in the present disclosure, thereby avoiding the loss of equipment and instruments for long-time production and reducing the cost.

3. Nitrogenous toxic gases such as NO and NO₂ are produced in chemical production (At present, China has few factories with the ability to treat nitrogen oxides), and greenhouse gases, e.g. N₂O, which is more serious than CO₂, will also be produced. However, there is no polluted gas and intermediate products during the process in the present disclosure.

4. Different byproducts are produced in the chemical method, but no additional by-products are produced in the method of the present disclosure.

B: Compared with the Reported Biological Fermentation Method:

1. Metabolic engineering methods require different designs and modifications of strains for different products each time. While in the present disclosure, different substrates may be combined for different products, which is more flexible.

2. At present, the yield of pimelic acid or octanedioic acid produced by fermentation is very low.

The present disclosure may realize the efficient production of various diacids (including glutaric acid, adipic acid, pimelic acid, suberic acid, C10, C12, C15 and other diacids) by using resting cell catalysis.

3. The problem that the product is difficult to purify in the later stage due to different metabolites produced by fermentation medium and cell metabolism is avoided.

C: Compared with the Reported Enzymatic Method:

1. Cumbersome steps such as cell disruption and enzyme purification may be avoided by using whole-cells as catalysts.

2. Cell catalysts are easier to be prepared and stored on a large scale compared with enzyme liquid catalysts.

### Other Advantages

1. The substrate is flexible, and different substrates may be used to produce a certain dicarboxylic acid.

2. The method in the disclosure can be widely used to produce various dicarboxylic acids such as C5, C6, C7, C8, C10, C12 and C15, wherein the enzyme and the reaction are the same.

3. The purification of the product is simple, which may be obtained by simple extraction and rotary evaporation.

4. Substrates that have not reacted completely may be recycled for the next recycling.

### BRIEF DESCRIPTION OF THE DRAWINGS

FIG. 1 shows a flow chart of the technical idea of the present disclosure;

FIG. 2 shows the structure and data related to plasmids screened by module I;

FIG. 3 shows the structure and data related to plasmids screened by module II;

FIG. 4 shows the structure and data related to plasmids screened by module III;

FIG. 5 is a data line chart of Example 4;

FIG. 6 is a data line chart of Example 5;

FIG. 7 is a data line chart of Example 7;

FIG. 8 is a data line chart of Example 8;

FIG. 9 is a GC-MS spectrum of the silanization product derivatized from glutaric acid;

FIG. 10 is a GC-MS spectrum of the silanization product derivatized from adipic acid;

FIG. 11 is a GC-MS spectrum of the silanization product derivatized from pimelic acid;

FIG. 12 is a GC-MS spectrum of the silanization product derivatized from suberic acid;

FIG. 13 shows a ¹H NMR spectrum of glutaric acid;

FIG. 14 shows a ¹H NMR spectrum of adipic acid;

FIG. 15 shows a ¹H NMR spectrum of pimelic acid;

FIG. 16 shows a ¹H NMR spectrum of suberic acid.

### DETAILED DESCRIPTION OF THE EMBODIMENTS

In order to make the purpose, technical scheme, and advantages of the present disclosure clearer, the present disclosure will be further described in detail below in combination with examples. The equipment and reagents used in the embodiments and examples can be obtained from commercial sources unless otherwise specified. The specific embodiments/examples described herein are only used to explain the present disclosure, but not to limit the present disclosure.

The proteins or fragments thereof involved in the present disclosure may be recombinant, natural, synthetic proteins or fragments thereof, the proteins or fragments thereof involved in the present disclosure may be natural purified products, or chemically synthesized products, or produced from prokaryotic or eukaryotic hosts (for example, bacteria, yeast, plants) using recombination technology.

According to the hosts used in the recombinant production scheme, the proteins of the present disclosure may be glycosylated or non-glycosylated.

The term “fragment” in the present disclosure refers to a polypeptide that basically maintains the same biological function or activity as the protein involved in the present disclosure. In view of the prior art in the field and the teachings of the present disclosure, it is not difficult for those skilled in the art to obtain active fragments of the protein according to the present disclosure. For example, in the disclosure, the biologically active fragment of “alcohol dehydrogenase” refers to fragment of “alcohol dehydrogenase”, but it can still maintain the full or partial functions of the full-length “alcohol dehydrogenase”. Generally, the biologically active fragments maintain at least 50% activity of the full-length “alcohol dehydrogenase”. Under more preferred conditions, the active fragment can maintain 60%, 70%, 80%, 90%, 95%, 99%, or 100% activity of the full-length “alcohol dehydrogenase”.

Eight enzymes are involved in the whole-cell synthesis pathway of dicarboxylic acid proposed in the present disclosure. Based on the prior art knowledge, it is not difficult for those of ordinary skill in the art to know that the eight enzymes involved in the present disclosure are not limited to the ones from specific source mentioned in the examples. Each enzyme can be replaced with enzymes from different sources with the same or similar catalytic functions, or a variant form of one or more of the eight specific source enzymes in the example can be used. The variant form has the same or similar function as one of the eight enzymes, the amino acid sequence of which is slightly different from that provided by the present disclosure. These variant forms include but are not limited to: the deletion, insertion and/or substitution of one or more (usually 1-30, preferably 1-10, more preferably 1-6, further preferably 1-3) amino acids, and the addition of one or more (usually within 20, preferably within 10, more preferably within 6 or 3) amino acids at the C-terminus and/or N-terminus. For example, it is well known to those skilled in the art that substitutions with amino acids with close or similar properties (such as isoleucine and leucine are substituted with each other) does not alter the function of the resulting protein. For another example, adding one or more amino acids at the C-terminus and/or N-terminus, such as adding a tag (for example a 6×His tag) to facilitate separation, usually does not change the function of the resulting protein.

In view of the teachings of the present disclosure and the prior art, those skilled in the art can perform amino acids substitutions as shown in the following table to produce mutants of conservative variation.

| | Representative | Preferred |
|---|---|---|
| Initial | substituted | substitution |
| residues | residues | residues |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The host cells of the present disclosure may be prokaryotic cells, such as bacterial cells; or lower eukaryotic cells, such as yeast cells. In specific embodiments, the strains include but are not limited to: Escherichia coli (E. coli), Corynebacterium glutamicum, Bacillus subtilis, Brevibacterium flavum, Serratia marcescens, and Saccharomyces cerevisiae. In a preferred embodiment, the strain is Escherichia coli (E. coli), which is also described in detail as an example in the present disclosure.

Host cells can be transformed with recombinant DNA by conventional techniques well known to those skilled in the art. When the host is a prokaryotic organism such as Escherichia co/i, competent cells that can absorb DNA can be harvested after the exponential growth phase, and then the competent cells were treated by CaCl₂ method, of which the steps used are well known in the art. Another method of transformation is to use MgCl₂. Transformation can also be performed by electroporation if necessary. When the host is a eukaryote, the following DNA transfection methods can be used: calcium phosphate co-precipitation method and conventional mechanical method such as microinjection, electroporation, liposome packaging, etc.

Based on the teachings of the present disclosure and the prior art, those skilled in the art can also understand that the recombinant cells of the present disclosure can be made into immobilized cells and other forms of utilization.

In the present disclosure, the term “expression vector” refers to bacterial plasmids, bacteriophages, yeast plasmids, plant cell viruses, mammalian cell viruses or other vectors well known in the art. In a word, any plasmid/vector can be used as long as they can replicate and stabilize in the host. An important feature of expression vectors is that they usually contain replication origins, promoters, marker genes, and translation control elements.

Those skilled in the art can use well-known methods to construct expression vectors containing DNA sequences encoded by exogenous enzymes and appropriate transcription/translation control signals, including in vitro recombinant DNA technology, DNA synthesis technology, and in vivo recombination technology. The DNA sequences can be effectively linked to appropriate promoters in the expression vectors to guide mRNA synthesis. The expression vectors also include ribosome binding site for translation initiation and transcription terminator.

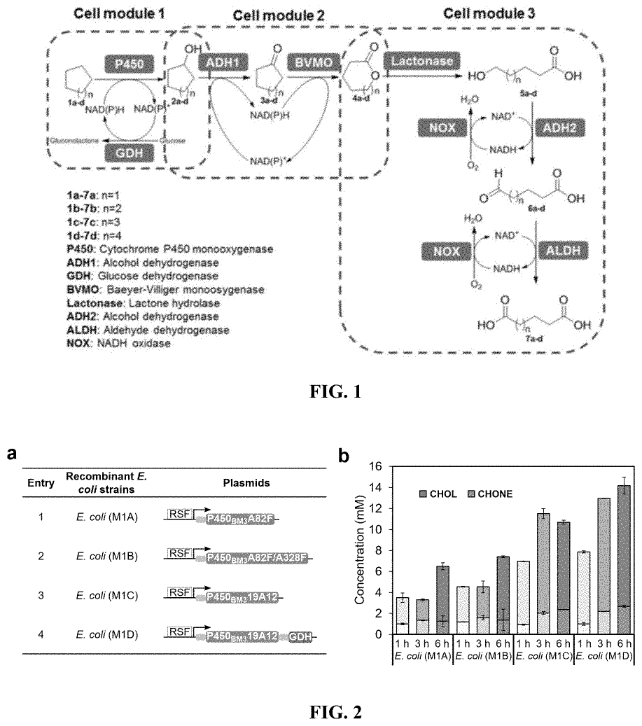

The purpose of the present disclosure is to propose a whole-cell biosynthetic pathway of α, ω-dicarboxylic acid (DCA), including eight enzymes and a six-step reaction. The method has been divided into three modules according to the reaction type (oxidation/reduction) or cofactor regeneration system. Taking the biological preparation pathway of adipic acid as an example, the function of module I: activating inert carbon-hydrogen bonds to realize the conversion of cyclohexane to cyclohexanol; the function of module II: realizing the conversion of cyclohexanol/cyclohexanone to F-caprolactone; the function of module III: realizing the conversion of caprolactone to adipic acid. Two systems are used in the present disclosure. One is to produce dicarboxylic acids using single-cell containing three modules as catalyst, and the other is to use a combination of multiple cells, that is, each module is expressed in a different host cell, followed by combination of them. A multicellular biocatalytic system (MBS) for the conversion of cycloalkanes into DCA requires only water and oxygen is formed. At the same time, each module can be separately used as a class of reactions to obtain DCA from different types of substrates (cycloalkanols, cycloalkanones, lactones). The idea of the present disclosure can be expressed by FIG. 1.

The present disclosure is specifically illustrated by taking the production reaction of adipic acid as an example. The specific contents of the disclosure are shown in the following examples.

### Example 1 Construction of Recombinant Plasmid

In this example, the genes of each enzyme were amplified and encoded by PCR using primers containing homologous arms, and the vector plasmids were amplified by PCR for linearization. Subsequently, different genes and linearized vectors formed 15 bp or 20 bp sticky terminus under the action of T5 exonuclease and were mixed. The details are as follows:

1. Construction of MID (pRSFDuet-1-P450_{BM3}19A12-GDH) with Recombinant Plasmid of Module I

Module I is the key step for activating inert carbon hydrogen bonds to produce corresponding alcohols, which is very challenging for the hydroxylation of small molecular cycloalkanes. In order to solve this problem, P450 BM3 mutants (A82F, A82F/A328F, 19A12) from Bacillus megaterium were used for exploration and comparison in this example. The related plasmid structure and data are shown in FIG. 2. According to the expression effect of the target products, 19A12 and GDH were finally selected to construct the recombinant strain E. coli (MID) to realize the hydroxylation of cyclohexane, and to add glucose to realize the construction of the cofactor circulation system.

The detailed construction process is as follows:

Linearization vector pRSFDuet-1
Primer:
SEQ ID NO. 1
F: GCCAGGATCCGAATTCGAGCTC,;
SEQ ID NO. 2
R: GTGGTGATGATGGTGATGGCTGCTG,;

The PCR amplification system (50 μL) was as follows: template pRSFDuet-1, plasmid 0.5-20 ng, each for a pair of mutation primers 1 μL (10 μM), Prime STAR Max DNA polymerase 25 μL, adding sterilized distilled water to 50 μL.

The PCR amplification procedure was as follows: (1) denaturing at 98° C. for 3 min, (2) denaturing at 98° C. for 30 s, (3) annealing at 55° C. for 15 s, (4) extending at 72° C. for 60 s, repeating steps (2)-(4) for 34 cycles, and finally extending at 72° C. for 5 min, and the products were preserved at 12° C.

P450_{BM3}19A12 gene was amplified. The sequence of the gene was shown in Yu H L, et al. Bioamination of alkane with ammonium by an artificially designed multienzyme cascade. Metab. Eng. 47, 184-189 (2018).

Primer:
19A12_homologous seq-Fwd:
SEQ ID NO. 3
CACCATCATCACCACGCAATTAAAGAAATGCCTCAGCCAAAAAC
G,;
19A12_RBS-Rev:
SEQ ID NO. 4
GATATATCTCCTTAGGTACCTTACCCAGCCCACACGTCTTTTGC,;

The PCR amplification system and procedure were the same as above, wherein, the template in the system was pET28a-19A12 plasmid (the plasmid could be obtained by introducing the synthesized 19A12 gene fragment into the plasmid pET28a according to the conventional method, and the method for obtaining the templates in other PCR systems were the same). The gene fragments could also be synthesized directly by a third-party company based on the base sequence.

The amplification of GDH genes, wherein the GDH genes were derived from Bacillus megaterium and the codon was optimized. The sequence was set forth in SEQ ID NO. 27:

Primer:
RBS_GDH-Fwd:
SEQ ID NO. 5
GGTACCTaaggagATATATCatgTATACAGATTTAAAAGATAAAGT
AGTAGTAATTACAGGTGGATC,;
R:
SEQ ID NO. 6
GCTCGAATTCGGATCCTGGCTTATCCGCGTCCTGCTTGGAATG,;

The PCR amplification system was the same as above, wherein, the template in the system was pET28a-GDH plasmid. The gene fragments could also be synthesized directly by a third-party company based on the base sequence. The PCR amplification procedure was as follows: (1) denaturing at 98° C. for 3 min, (2) denaturing at 98° C. for 10 s, (3) annealing at 55° C. for 15 s, (4) extending at 72° C. for 10 s, repeating steps (2)-(4) for 30 cycles, and finally extending at 72° C. for 5 min, and the products were preserved at 12° C.

Nucleic acid electrophoresis was used to detect whether the target band was obtained, and the remaining templates in the PCR products were digested with Dpn I after confirming the band was obtained. The system (50 μL) was: 5 μL CutSmart Buffer, 2 μL Dpn I, 43 μL PCR products. The templates were digested at 37° C. for 5 h, and then the products after digestion were inactivated at 80° C. for 15 min. The gels were recovered with OMEGA recovery kit.

The linearized vectors and PCR amplified gene fragments were joined together by forming 15 bp or 20 bp sticky terminus under the action of T5 exonuclease. The specific method was as follows: the target fragments and linearized vectors (the amount of linearized vectors was controlled to be 30-50 ng) were added into a 5 μL reaction system at a molar ratio of 3:3:1, then T5 exonuclease and buffer 4.0 were added, adding water if the reaction system was less than 5 μL. After adding T5 exonuclease for 5 minutes, and 50 μL of DH5α competent cells were added immediately to transform according to the basic steps of conventional transformation, after the culture medium was added to resuscitate for 1 hour, the mixture was transferred to LB solid medium containing the corresponding Kan resistance (50 μg/mL) and cultured overnight, and the corresponding transformants were taken and sent to the sequencing company for DNA sequencing. Finally, the correct recombinant MID (pRSFDuet-1-P450_{BM3}19A12-GDH) was obtained.

2. Construction of M2E (pRSFDuet-1-ADH1-BVMO) with Recombinant Plasmid of Module II

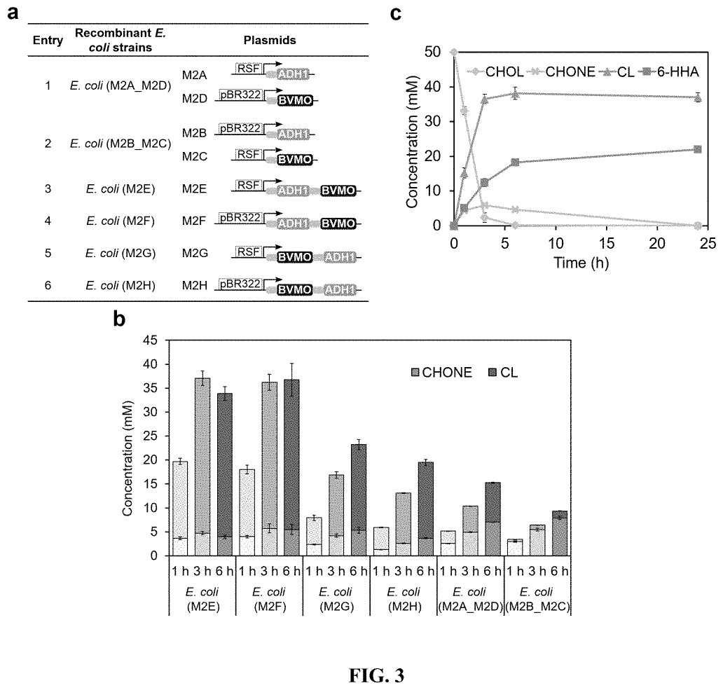

In order to realize the catalytic process of the intermediate step from cyclohexanol or cyclohexanone to F-caprolactone, a second module was designed in this example, and six types of recombinant cells were constructed. The recombinant cells included alcohol dehydrogenase (ADH1, Lactobacillus brevis ATCC 14869) and Baeyer-Villiger monooxygenase (BVMO, Acinetobacter sp. NCIMB 9871, also containing double mutation sites C376I/M400I), which were necessary for the catalytic reaction. The related plasmid structure and data are shown in FIG. 3, wherein E. coli (M2E) showed the best catalytic performance, that is, 37 mM F-caprolactone could be detected to be produced within 6 hours with 50 mM cyclohexanol as the substrate, and at the same time, 6-hydroxycaprolic acid could be detected due to the spontaneous hydrolysis of F-caprolactone in buffer. Finally, E. coli (M2E) was selected for the next combination.

The processing method of linearized vector pRSFDuet-1 was the same as step 1.

The amplification of ADH1 genes, where the ADH1 genes were derived from Acinetobacter sp. NCIMB9871 and the codon was optimized. The sequence was set forth in SEQ ID NO. 28:

Primer:
ADH1_homologous seq-Fwd:
SEQ ID NO. 7
CACCATCATCACCACATGAGCAATCGTCTGGATGGTAAAGTTG,;
ADH1_RBS-Rev:
SEQ ID NO. 8
GATATATctccttAGGTACCTTACTGTGCGGTATAACCACCATCCA
C,;

The PCR amplification system and procedure were the same as the GDH gene amplification conditions in Step 1, wherein, the template in the system was pRSFDuet-1-ADH1 plasmid. The gene fragments could also be synthesized directly by a third-party company based on the base sequence.

The amplification of BVMO genes, where the BVMO genes were derived from Acinetobacter sp. NCIMB9871 and the codon was optimized. The sequence was set forth in SEQ ID NO. 29;

Primer:
RBS_BVMO-Fwd:
SEQ ID NO. 9
GGTACCTaaggagATATATCatgtcacaaaaaatggattttgatgc
tatcgtg,;
BVMO_homologous seq-Rev:
SEQ ID NO. 10
GAATTCGGATCCTGGCttaggcattggcaggttgcttgatatc,;

The PCR amplification system was the same as above, wherein, the template in the system was pET28a-BVMO plasmid. The gene fragments could also be synthesized directly by a third-party company based on the base sequence. The PCR amplification procedure was as follows: (1) denaturing at 98° C. for 3 min, (2) denaturing at 98° C. for 10 s, (3) annealing at 55° C. for 15 s, (4) extending at 72° C. for 15 s, repeating steps (2)-(4) for 30 cycles, and finally extending at 72° C. for 5 min, and the products were preserved at 12° C.

Nucleic acid electrophoresis was used for detection and recovery, the operation was the same as above.

The linearized vectors and PCR amplified gene fragments were joined together by forming 15 bp or 20 bp sticky terminus under the action of T5 exonuclease. The specific method was the same as above, and the correct recombinant M2E (pRSFDuet-1-ADH1-BVMO) was obtained by DNA sequencing.

3. Construction of M3B (pETDuet-1-ADH2-ALDH), M3E (pRSFDuet-1-Lactonase-NOX) and M3J (pETDuet-1-ADH2-ALDH-Lactonase-NOX) with Recombinant Plasmid of Module III.

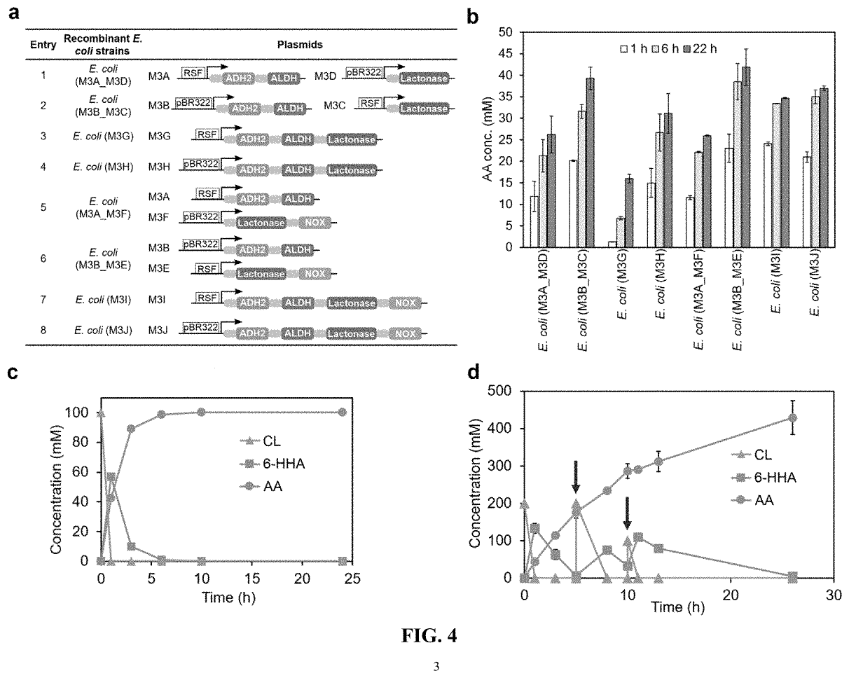

In order to realize the catalytic process from F-caprolactone to adipic acid, a third module was designed in this example, and eight types of recombinant cells were constructed. The recombinant cells included the necessary lactonase (lactonase, Rhodococcus sp. HI-31), alcohol dehydrogenase (ADH2, Acinetobacter sp. NCIMB9871), aldehyde dehydrogenase (ALDH, Acinetobacter sp. NCIMB9871), and NADH oxidase (NOX, Lactobacillus brevis DSM 20054) was added under certain conditions to improve the circulation efficiency of NAD⁺. The related structure and data are shown in FIG. 4. The eight reconstituted cells are identified to have the effect of catalyzing the formation of adipic acid from F-caprolactone, wherein E. coli (M3B_M3E) showed the best productivity, i.e. 42 mM adipic acid (50 mM substrate) was produced within 22 hours. When the reaction process was further studied with a substrate concentration of 100 mM, it was found that the substrate could be completely converted into adipic acid within 6 hours without the accumulation of intermediate products. However, with the increasing of the substrate concentration and the decreasing of pH, the reaction could not continue to proceed efficiently. Therefore, with the strategy of feeding batch and adjusting pH in the reaction, a total of 450 mM adipic acid could be obtained with little accumulation of intermediate products, after adding 500 mM substrates for a total of three feeding batches for 26 hours. Finally, E. coli (M3B_M3E) was selected for the next combination.

The vector pRSFDuet-1, pETDuet-1 were linearized, the processing method was the same as step 1.

The amplification of ADH2 genes, wherein the ADH2 genes were derived from Acinetobacter sp. NCIMB9871, and the codon was optimized. The sequence was set forth in SEQ ID NO. 30:

Primer:
ADH2_homologous seq-Fwd:
SEQ ID NO. 11
GCCATCACCATCATCACCACCATTGTTATTGCGTTACCCATCATG
G,;
ADH2_RBS-Rev:
SEQ ID NO. 12
GATATATctccttAGGTACCTTAGTTCTCGTGCATCAGAACGATAC
G,;

The PCR amplification system was the same as above, wherein, the template in the system was pRSFDuet-1-ADH2 plasmid. The gene fragments could also be synthesized directly by a third-party company based on the base sequence. The PCR amplification procedure were the same as the GDH gene amplification conditions in step 1.

The amplification of ALDH genes, where the ALDH genes were derived from Acinetobacter sp. NCIMB9871, and the codon was optimized. The sequence was set forth in SEQ ID NO. 31:

Primer:
RBS_ALDH-Fwd:
SEQ ID NO. 13
GGTACCTaaggagATATATCATGAACTATCCGAATATTCCGCTGTA
TATTAACG,;
ALDH_homologous seq-Rev:
SEQ ID NO. 14
GCTCGAATTCGGATCCTGGCTTAGTTCAGCTGGGTGATAAATTTGG
TG,;

The PCR amplification system was the same as above, wherein, the template in the system was pETDuet-1-ALDH plasmid. The gene fragments could also be synthesized directly by a third-party company based on the base sequence. The PCR amplification procedure was as follows: (1) denaturing at 98° C. for 3 min, (2) denaturing at 98° C. for 10 s, (3) annealing at 55° C. for 15 s, (4) extending at 72° C. for 15 s, repeating steps (2)-(4) for 30 cycles, and finally extending at 72° C. for 5 min, and the products were preserved at 12° C.

The amplification of Lactonase genes, where the Lactonase genes were derived from Rhodococcus sp. HI-31, and the codon was optimized. The sequence was set forth in SEQ ID NO. 32:

Primer:
Lactonase_homologous seq-Fwd:
SEQ ID NO. 15
GCCATCACCATCATCACCACACCAATATTAGCGAAACCCTGAGCA
C,;
Lactonase_RBS-Rev:
SEQ ID NO. 16
GATATATctccttAGGTACCTTATTCCAGGGCTTTCTGATACCATG
CTG,;

The PCR amplification system was the same as above, wherein, the template in the system was pRSFDuet-1-Lactonase plasmid. The gene fragments could also be synthesized directly by a third-party company based on the base sequence. The PCR amplification procedure were the same as the GDH gene amplification conditions in step 1.

The amplification of NOX genes, the sequence was set forth in SEQ ID NO. 33:

Primer:
RBS_NOX-Fwd:
SEQ ID NO. 17
GGTACCTaaggagATATATCATGAAAGTTATCGTAATTGGTTGTAC
TCATGCCG,;
NOX_homologous seq-Rev:
SEQ ID NO. 18
GCTCGAATTCGGATCCTGGCTTATTCCGTCACTTTTTCAGCCGCAT
GAG,;

The PCR amplification system was the same as above, wherein, the template in the system was pRSFDuet-1-NOX plasmid. The gene fragments could also be synthesized directly by a third-party company based on the base sequence. The PCR amplification procedure was as follows: (1) denaturing at 98° C. for 3 min, (2) denaturing at 98° C. for 10 s, (3) annealing at 55° C. for 15 s, (4) extending at 72° C. for 20 s, repeating steps (2)-(4) for 30 cycles, and finally extending at 72° C. for 5 min, and the products were preserved at 12° C.

Nucleic acid electrophoresis was used for detection and recovery.

The linearized vectors and PCR amplified gene fragments were joined by forming 15 bp or 20 bp sticky terminus under the action of T5 exonuclease. The specific method was as follows: the target fragments and linearized vectors (the amount of linearized vector were controlled to be 30-50 ng) were added into a 5 μL reaction system at a molar ratio of 3:3:1 (ADH2:ALDH:pETDuet-1=3:3:1, Lactonase:NOX:pRSFDuet-1=3:3:1), and then T5 exonuclease and buffer 4.0 were added, adding water if the reaction system was less than 5 μL. T5 exonuclease was added for 5 min, then 50 μL of DH5a competent cells were added immediately to transform according to the basic steps of conventional transformation. After the culture medium was added to resuscitate for 1 hour, the mixture was transferred to LB solid medium containing the corresponding resistance Amp (100 μg/mL) or Kan (50 μg/mL) and cultured overnight, and the corresponding transformants were taken and sent to the sequencing company for DNA sequencing, and finally the correct recombinant was obtained. M3B (pETDuet-1-ADH2-ALDH), M3E (pRSFDuet-1-Lactonase-NOX) were obtained.

Linearized M3B (pETDuet-1-ADH2-ALDH)
Primer:
F:
SEQ ID NO. 19
GCCAGGATCCGAATTCGAGCTC,;
ALDH_RBS-Rev:
SEQ ID NO. 20
GATATATctccttAGGTACCTTAGTTCAGCTGGGTGATAAATTTGG
TG,;

The PCR amplification system was the same as above, wherein, the template in the system was pETDuet-1-ADH2-ALDH plasmid. The gene fragments could also be synthesized directly by a third-party company based on the base sequence. The PCR amplification procedure was as follows: (1) denaturing at 98° C. for 3 min, (2) denaturing at 98° C. for 10 s, (3) annealing at 55° C. for 15 s, (4) extending at 72° C. for 2 min, repeating steps (2)-(4) for 30 cycles, and finally extending at 72° C. for 5 min, and the products were preserved at 12° C.

The amplification of Lactonase-NOX genes

Primer:
RBS_Lactonase-Fwd:
SEQ ID NO. 21
GGTACCTaaggagATATATCATGACCAATATTAGCGAAACCCTGAG
C,;
NOX_homologous seq-Rev:
SEQ ID NO. 22
GCTCGAATTCGGATCCTGGCTTATTCCGTCACTTTTTCAGCCGCAT
GAG,;

The PCR amplification system was the same as above, wherein, the template in the system was pRSF-Duet-1-Lactonase-NOX plasmid. The gene fragments could also be synthesized directly by a third-party company based on the base sequence. The PCR amplification procedure was as follows: (1) denaturing at 98° C. for 3 min, (2) denaturing at 98° C. for 10 s, (3) annealing at 55° C. for 15 s, (4) extending at 72° C. for 30 s, repeating steps (2)-(4) for 30 cycles, and finally extending at 72° C. for 5 min, and the products were preserved at 12° C.

Nucleic acid electrophoresis was used for detection and recovery.

The linearized vectors and PCR amplified gene fragments were joined together by forming 15 bp or 20 bp sticky terminus under the action of T5 exonuclease. The specific method was as follows: the target fragments Lactonase-NOX and linearized M3B (the amount of linearized vectors was controlled to be 30-50 ng) were added into a 5 μL reaction system, the follow-up operation was the same as step 1. The correct recombinant M3J (pETDuet-1-ADH2-ALDH-Lactonase-NOX) was finally obtained by DNA sequencing.

4. Construction of Recombinant Plasmid (M12A) of Module I and II

Linearized M1D (pRSFDuet-1-P450_{Bm3}19A12-GDH)
Primer:
F:
SEQ ID NO. 23
GCCAGGATCCGAATTCGAGCTC,;
GDH_RBS-Rev:
SEQ ID NO. 24
GATATATCTCCTTAGGTACCTTATCCGCGTCCTGCTTGGAATG,;

The PCR amplification system was the same as above, wherein, the template in the system was pRSFDuet-1-P450_{BM3}19A12-GDH plasmid. The gene fragments could also be synthesized directly by a third-party company based on the base sequence. The PCR amplification procedure was the same as the conditions in linearized M3B.

Amplified gene ADH1-BVMO
Primer:
RBS_ADH1-Fwd:
SEQ ID NO. 25
GGTACCTaaggagATATATCATGAGCAATCGTCTGGATGGTAAAGT
TG,;
BVMO_homologous seq-Rev:
SEQ ID NO. 26
GAATTCGGATCCTGGCttaggcattggcaggttgcttgatatc,;

The PCR amplification system was the same as above, wherein, the template in the system was pRSFDuet-1-P450_{BM3}19A12-GDH plasmid. The gene fragments could also be synthesized directly by a third-party company based on the base sequence. The PCR amplification procedure was as follows: (1) denaturing at 98° C. for 3 min, (2) denaturing at 98° C. for 10 s, (3) annealing at 55° C. for 15 s, (4) extending at 72° C. for 30 s, repeating steps (2)-(4) for 30 cycles, and finally extending at 72° C. for 5 min, and the products were preserved at 12° C.

Nucleic acid electrophoresis was used for detection and recovery.

The linearized vectors and PCR amplified gene fragments were joined by forming 15 bp or 20 bp sticky terminus under the action of T5 exonuclease. The specific method was as follows: the target fragments ADH1-BVMO and linearized MID (the amount of linearized vector was controlled to be 30-50 ng) were added into a 5 μL reaction system at a molar ratio of 3:3:1, the follow-up operation was the same as step 1. The correct recombinant M12A (pRSFDuet-1-P450_{BM3}19A12-GDH-ADH1-BVMO) was finally obtained by DNA sequencing.

### Example 2 Construction of Recombinant Cells

The host cell of the present disclosure may be a prokaryotic cell or a lower eukaryotic cell as described above, and in this example, E. coli was taken as an example for specific description.

1. E. coli (M3B_M3E)

The two recombinant plasmids, M3B and M3E, were transformed into E. coli BL21 (DE3) by the general electrotransformation method at a molar ratio of 1:1, and cultured on LB solid culture containing two resistant Amp (100 μg/mL) and Kan (50 μg/mL) to obtain the recombinant cells E. coli (M3B_M3E) that could express the enzymes involved in module III alone.

2. E. coli (M2E)

The recombinant plasmid M2E was transformed into E. coli BL21(DE3) and cultured on LB solid culture containing Kan (50 μg/mL) to obtain recombinant cells E. coli (M2E) that could express the enzymes involved in module II alone.

3. E. coli (MID)

The recombinant plasmid MID was transformed into E. coli BL21(DE3) and cultured on LB solid culture containing Kan (50 μg/mL) to obtain recombinant cells E. coli (MID) that could express the enzymes involved in module I alone.

4. E. coli (M2E_M3J)

The two recombinant plasmids, M2E and M3J, were transformed into E. coli BL21 (DE3) at a molar ratio of 1:1 and cultured on LB solid culture containing two resistant Amp (100 μg/mL) and Kan (50 μg/mL) to obtain recombinant cells E. coli (M2E_M3J) that could simultaneously express the enzymes involved in modules II and III.

5. E. coli (M12A_M3J)

The two recombinant plasmids, M12A and M3J, were transformed into E. coli BL21 (DE3) at a molar ratio of 1:1 and cultured on LB solid culture containing two resistant Amp (100 μg/mL) and Kan (50 μg/mL) to obtain recombinant cells E. coli (M12A_M3J) that could simultaneously express the enzymes involved in modules I, II and III.

### Example 3 Protein Expression and Preparation of Whole-Cell Catalyst

The constructed recombinant cells were inoculated into 3 mL of LB liquid medium containing the corresponding resistance and cultured at 37° C. and 220 rpm for about 6 h, and 1 mL of the bacterial culture medium was transferred to 50 mL of TB culture medium containing the corresponding resistance and cultured at 37° C. and 220 rpm for about 2-3 h until OD₆₀₀=0.6-0.8. IPTG was added to make the final concentration 0.2 mM. The temperature was then adjusted to 25° C. and the recombinant cells were induced for 14-16 h. The cell induction conditions containing module III were adjusted to a final concentration of IPTG of 0.1 mM and induced at 20° C. for 20 h.

The bacterium was centrifuged at 15° C. and 3040×g for 10 min to collect the bacterium after induction, and the bacterium was washed with 200 mM potassium phosphate buffer with pH 8.0 after collection, and the cells were used as catalyst for subsequent reaction.

The recombinant cells prepared by the present disclosure can be applied to the catalysis and conversion of various types (including C5, C6, C7, C8, C10, C12, C15, etc.) cycloalkanes, cyclitols and lactones. The applications in examples 4-9 will be described in detail in the following.

### Example 4 Biotransformation of ε-Caprolactone to Adipic Acid

The recombinant cell E. coli (M3B_M3E) expressing module III was suspended in 200 mM potassium phosphate buffer (pH 8.0) to an OD₆₀₀ of 40. 4 mL of the suspension was taken into a 100 mL reaction flask and caprolactone was added to start the reaction. The reaction conditions were as follows: the reaction was carried out in a 100 mL (ground mouth with lid) conical flask at 30° C. and 220 rpm. In the reaction, the substrate was added in batches (caprolactone was added at the beginning of the reaction to make the final concentration of the substrate 200 mM, and 200 mM and 100 mM substrates were added when the reaction proceeded to 6 h and 10 h). During the reaction, 10 M NaOH was used to adjust the pH so that the reaction system was maintained at pH 8.0.

In order to detect the production of adipic acid and 6-hydroxycaproic acid at the set time point, 50 μL of the reaction solution was added with 450 μL of water and 50 μL of 4 M HCl, and then 500 μL of ethyl acetate was added for extraction with vigorous shaking, the mixture was centrifuged at 13680×g for 1 min, the upper organic phase was taken and dried by adding Na₂SO₄ for subsequent derivatization. In order to detect the production of caprolactone, 50 μL of the reaction solution was added with 450 μL of water and 500 μL of ethyl acetate (containing 2 mM n-decane as an internal standard) for extraction with vigorous shaking. The mixture was centrifuged at 13680×g for 1 min. The upper organic phase was taken and dried by adding Na₂SO₄ directly for GC analysis.

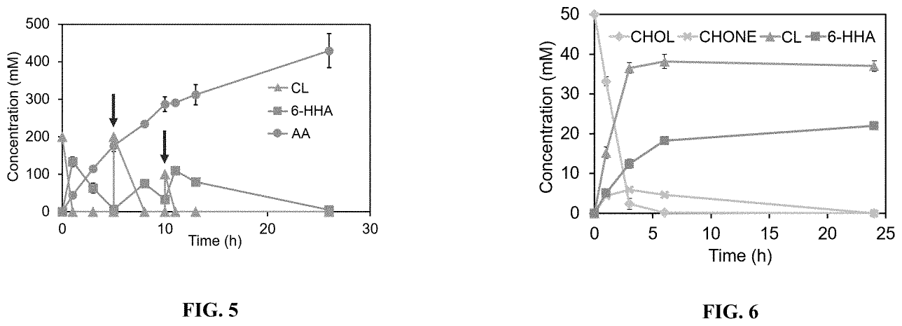

The third module was designed in this example, which included the necessary lactonase (Rhodococcus sp. HI-31), alcohol dehydrogenase (ADH2, Acinetobacter sp. NCIMB9871), aldehyde dehydrogenase (ALDH, Acinetobacter sp. NCIMB9871), and NADH oxidase (NOX, Lactobacillus brevis DSM 20054) was added under certain conditions to improve the circulation efficiency of NAD⁺. 42 mM adipic acid (50 mM substrate) was produced from E. coli (M3B_M3E) within 22 hours. When the reaction process was further studied with a substrate concentration of 100 mM, it was found that the substrate could be completely converted into adipic acid within 6 hours without the accumulation of intermediate products. However, with the increasing of the substrate concentration and the decreasing of pH, the reaction could not continue to proceed efficiently. Therefore this example adopts the strategy of feeding batch and adjusting pH in the reaction, a total of 450 mM adipic acid (i.e. 63 g/L adipic acid) could be obtained with little accumulation of intermediate products, after adding 500 mM of substrates for a total of three feeding batches for 26 hours. The data line chart of was shown in FIG. 5.

### Example 5 Biotransformation of Cyclohexanol to ε-Caprolactone

The recombinant cell E. coli (M2E) expressing module II was suspended in 100 mM of potassium phosphate buffer (pH 8.0) to an OD₆₀₀ of 20. 21.5 μL (final concentration of 50 mM) cyclohexanol was added to 4 mL reaction solution to start the reaction. The reaction was carried out at 25° C. and 220 rpm. In order to detect 6-hydroxycaproic acid produced by spontaneous hydrolysis at the set time point, 100 μL of reaction solution was added with 400 μL of water and 50 μL of 4 M HCL, and then 500 μL of ethyl acetate was added to shake vigorously for extraction, the mixture was centrifuged at 13680×g for 1 min, the upper organic phase was taken and dried by adding Na₂SO₄ for subsequent derivatization. In order to detect the amount of cyclohexanol, cyclohexanone and caprolactone, 100 μL of reaction solution was added with 400 μL of water and 500 μL of ethyl acetate (containing 2 mM n-decane as an internal standard) and vigorously shaken for extraction, the mixture was centrifuged at 13680×g for 1 min. The upper organic phase was taken and dried by adding Na₂SO₄ directly for GC analysis.

The second module was designed in this example. The second module included alcohol dehydrogenase (ADH1, Lactobacillus brevis ATCC 14869) and Baeyer-Villiger monooxygenase (BVMO, Acinetobacter sp. NCIMB9871, also containing double mutation sites C376I/M400I), which was necessary for the catalytic reaction. The catalytic effect of E. coli (M2E) was: 37 mM ε-caprolactone could be detected to be produced within 6 hours with 50 mM cyclohexanol as the substrate, and at the same time, 6-hydroxycaprolic acid could be detected due to the spontaneous hydrolysis of ε-caprolactone in buffer. The data line chart is shown in FIG. 6.

### Example 6 Biotransformation of Cyclohexane to Cyclohexanol

The recombinant cell E. coli (MID) expressing module I was suspended in 100 mM of potassium phosphate buffer (pH 8.0) to an OD₆₀₀ of 20. 0.05 g/L of glucose was added into the 4 mL reaction solution for cofactor circulation, and 22 μL of cyclohexane (final concentration of 50 mM) was added to react at 25° C. and 220 rpm. At the set time point, 100 μL of the reaction solution was diluted with 400 μL of water, and then 500 μL of ethyl acetate (containing 2 mM n-decane as an internal standard) was added for extraction with vigorous shaking, the mixture was centrifuged at 13680×g for 1 min. The upper organic phase was taken and dried by adding Na₂SO₄ directly for GC analysis.

The product was mainly cyclohexanol, which would also be partially converted to cyclohexanone, both of which were substrates for the next module.

### Example 7 Bioconversion of Cyclohexanol to α, ω-Dicarboxylic Acid

The present disclosure is not limited to the conversion of cyclohexanol to adipic acid but is also applicable to the conversion of other similar cycloalkanols or cycloalkanones to diacids.

Cycloalkanol (final concentration of 50 mM; 18.3 μL cyclopentanol, 21.5 μL cyclohexanol, 24.8 μL cycloheptanol or 28.4 μL cyclooctanol) was added to 4 mL E. coli MBS1 suspension (final OD₆₀₀ was 40, recombinant cells expressing module II E. coli (M2E) and module III E. coli (M3B_M3E) were suspended in potassium phosphate buffer (0.2 M, pH 8.0) at a ratio of 2:1) or E. coli (M2E_M3J) (final OD₆₀₀ was 40). The reaction was carried out in a 100 mL shake flask at 25° C. and 220 rpm. According to the method in Example 4, samples were taken at a set time for subsequent derivatization treatment and gas phase analysis directly.

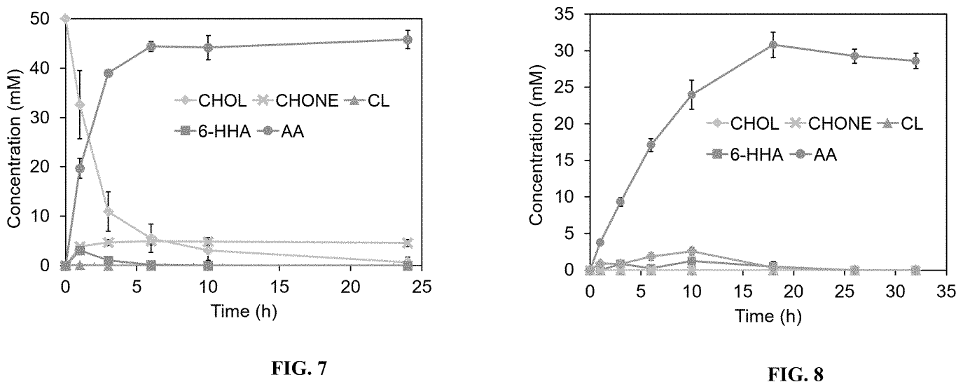

In this example, a single-cell system and a multi-cell combination system were designed to realize the conversion of cyclohexanol or cyclohexanone to adipic acid. The single-cell system E. coli (M2E_M3J) performed poorly, but the conversion from cyclohexanol to adipic acid could also be achieved, that is, 3 mM adipic acid could be obtained from 50 mM cyclohexanol without any optimization. Module II E. coli (M2E) and module III E. coli (M3B_M3E) were combined to form a multi-cell combination system MBS1. After experimental exploration, it was found that the best catalytic effect can be achieved when the ratio of the cell mass of module II to module III was 2:1 or 1:1. Considering the reaction cost, the present disclosure enumerated the reaction process when the total cell density was OD₆₀₀ of 40 and the ratio was 2:1. When the reaction reached 6 hours, 50 mM cyclohexanol could be converted to 46 mM adipic acid, only 4 mM intermediate product cyclohexanone remained. The data line chart was shown in FIG. 7.

The data for the production of α, ω-dicarboxylic acids from several other cycloalkanols are shown in the table below. 1: cycloalkane; 2: cyclitol; 3: cyclic ketone; 4: lactone; 5: hydroxy acid; 7: diacid. The a-d corresponds to the structural formulas in FIG. 1, and represents material represented by five-carbon, six-carbon, seven-carbon, and eight-carbon, respectively.

| | | Conc. of | | | | | |
|---|---|---|---|---|---|---|---|
| | | DCAs^{c} | Product distribution^{d} [%] | | | | |
| Entry | Substrate | (mM) | 2(a-d) | 3(a-d) | 4(a-d) | 5(a-d) | 7(a-d) |
| 1 | 2a^{a} | 48 | 0 | 7 | 0 | 0 | 93 |
| 2 | 2b^{a} | 46 | 0 | 8 | 0 | 0 | 92 |
| 3 | 2c^{a} | 49 | 0 | 0 | 0 | 0 | >99 |
| 4 | 2d^{a} | 42 | 0 | 0 | 0 | 7 | 93 |

^{a}Reactions were conducted with indicated substrates (50 mM) in 4 mL cell suspensions at 16 g CDW L⁻¹. EC2_3 containing E. coli (M2E) and E. coli (M3B_M3E) at a ratio of 2:1 was in 200 mM KP buffer (pH 8.0) at 25° C. and 200 rpm for 24 h.
^{c}Determined by gas chromatography.
^{d}Relative amounts based on the concentrations of dicarboxylic acids 7a-d and analyzed by gas chromatography.

### Example 8 Biotransformation of Cycloalkane to α, ω-Dicarboxylic Acid

The present disclosure is not limited to the conversion of cyclohexane to adipic acid but is also applicable to the conversion of other similar cycloalkanes to diacids.

44 μL of cyclohexane (final concentration was 100 mM) was added to 4 mL of E. coli MBS2 suspension (final OD₆₀₀ was 30, the ratio of recombinant cells expressing module I E. coli (MID), module II E. coli (M2E) and module III E. coli (M3B_M3E) was 2:1:2) resuspended in potassium phosphate buffer (0.2 M, pH 8.0) or E. coli (M12A_M3J) (final OD₆₀₀ was 30). The reaction was carried out in a 100 mL shake flask at 25° C. and 220 rpm. It is worthy to note that an additional 0.05 g/mL glucose needs to be added to this reaction system to promote the regeneration of NADPH, and the pH was maintained at about 8.0 by adding 10 M NaOH during the reaction. According to the method in Example 4, samples were taken at a set time for subsequent derivatization treatment and gas phase analysis directly.

When studying the conversion of other cycloalkanes into corresponding α, ω-dicarboxylic acids, the substrates were added so that the final concentration was 50 mM, that is, 19.5 μL cyclopentane, 22 μL cyclohexane, 24.7 μL cycloheptane and 27.5 μL cyclooctane were added respectively. Other reaction conditions were consistent with the above conditions.

The cell that reacted with cyclohexane was named E. coli (M12A_M3J), which contained module I, module II and module III, i.e. a single-cell system, 4 mM adipic acid could be obtained from 50 mM cyclohexane without any optimization, which proves that the conversion of cyclohexane to adipic acid could realize by using single cell. Module I E. coli (MID), module II E. coli (M2E) module III E. coli (M3B_M3E) combined to form a multi-cell combination system MBS2. After experimental exploration, it was found that the best catalytic effect can be achieved when the total cell density is OD₆₀₀ of 30, and when the ratio of the module I, module II and module III was 2:1:2. In this process, MBS2 could convert 100 mM cyclohexane into 32 mM adipic acid without the formation of intermediate products when the catalytic process was carried out for 20 hours. The unreacted cyclohexane could be recovered by simple ethyl acetate extraction, and adipic acid could be easily obtained by ethyl acetate extraction when the pH of the reaction solution was adjusted to about 2. The data line chart was shown in FIG. 8.

The data for the production of α, ω-dicarboxylic acids from several other cycloalkanols are shown in the table below. 1: cycloalkane; 2: cyclohexanol; 3: cyclone; 4: lactone; 5: hydroxy acid; 7: diacid. The a-d corresponds to the structural formula in FIG. 1, representing the substances represented by five-carbon, six-carbon, seven-carbon, and eight-carbon, respectively.

| | | Conc. of | | | | | |
|---|---|---|---|---|---|---|---|
| | | DCAs ^{c} | Product distribution^{d} [%] | | | | |
| Entry | Substrate | (mM) | 2(a-d) | 3(a-d) | 4(a-d) | 5(a-d) | 7(a-d) |
| 1 | 1a^{b} | 12.4 | 0 | 0 | 0 | 0 | >99 |
| 2 | 1b^{b} | 20.4 | 0 | 0 | 0 | 0 | >99 |
| 3 | 1c^{b} | 19.6 | 0 | 0 | 0 | 0 | >99 |
| 4 | 1d^{b} | 6.1 | 13 | 17 | 0 | 17 | 53 |

^{b}Reactions were conducted with indicated substrates (50 mM) in 4 mL cell suspension at 12 g CDW L⁻¹. EC1_2_3 containing E. coli (MIE), E. coli (M2E) and E. coli (M3B_M3E) at ratio 2:1:2 was in 200 mM KP buffer (pH 8.0) with 0.05 g mL⁻¹ glucose at 25° C. and 200 rpm for 24 h.
^{c} Determined by gas chromatography.
^{d}Relative amounts based on the concentrations of dicarboxylic acids 7a-d and analyzed by gas chromatography.

### Example 9 Preparation of Different α, ω-Dicarboxylic Acids

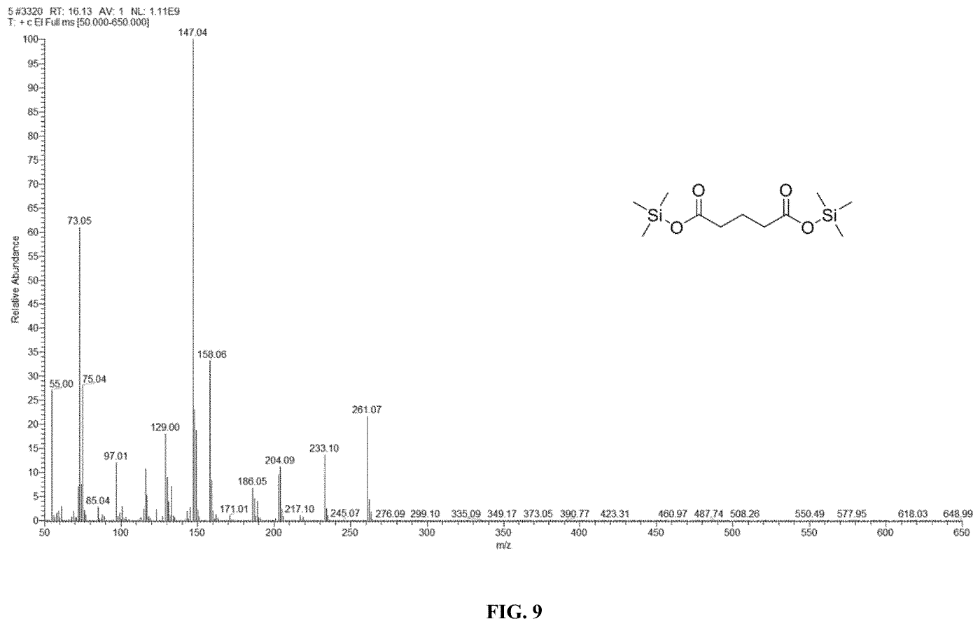

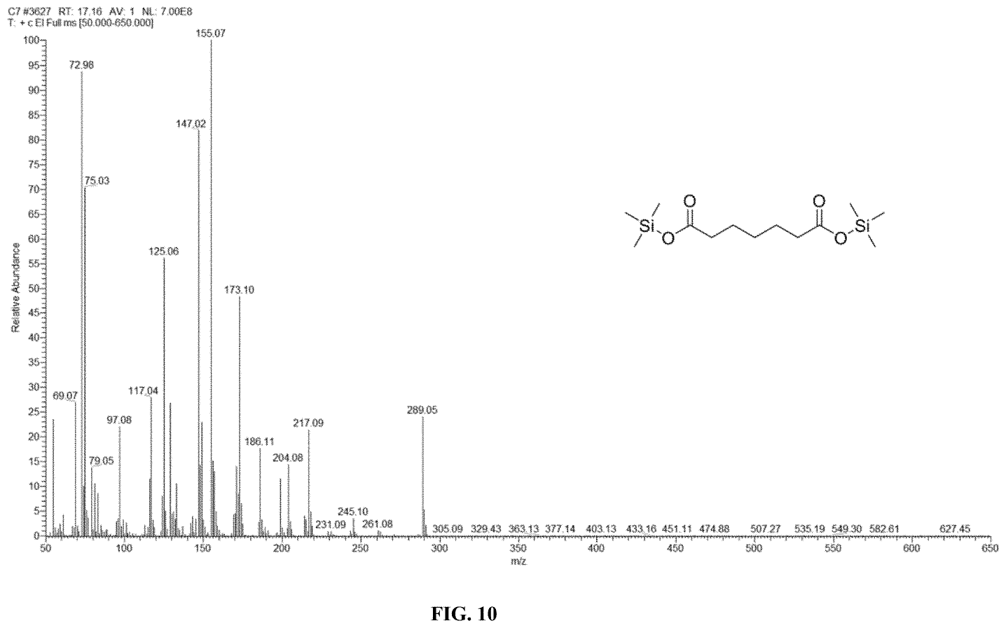

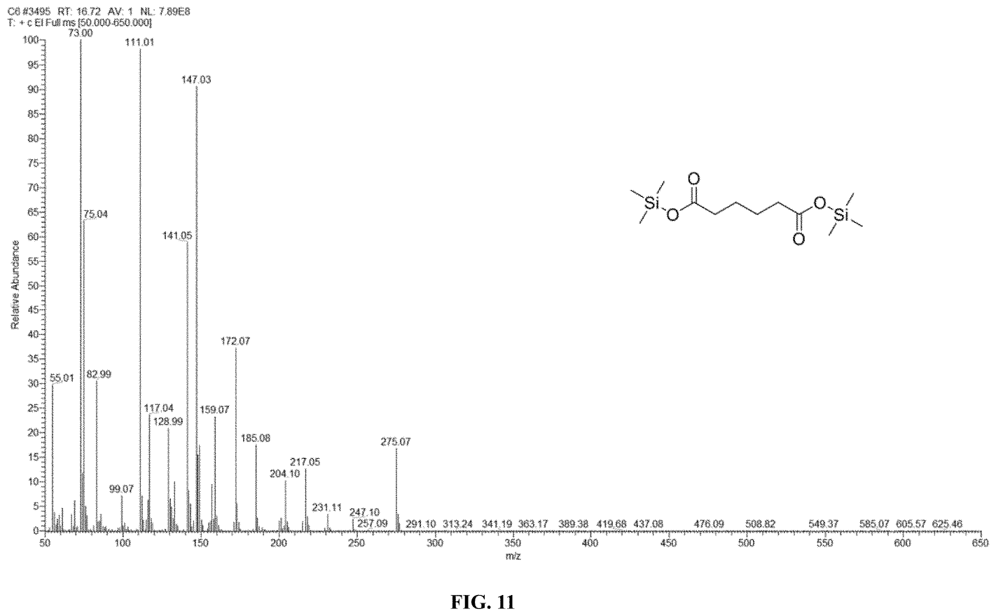

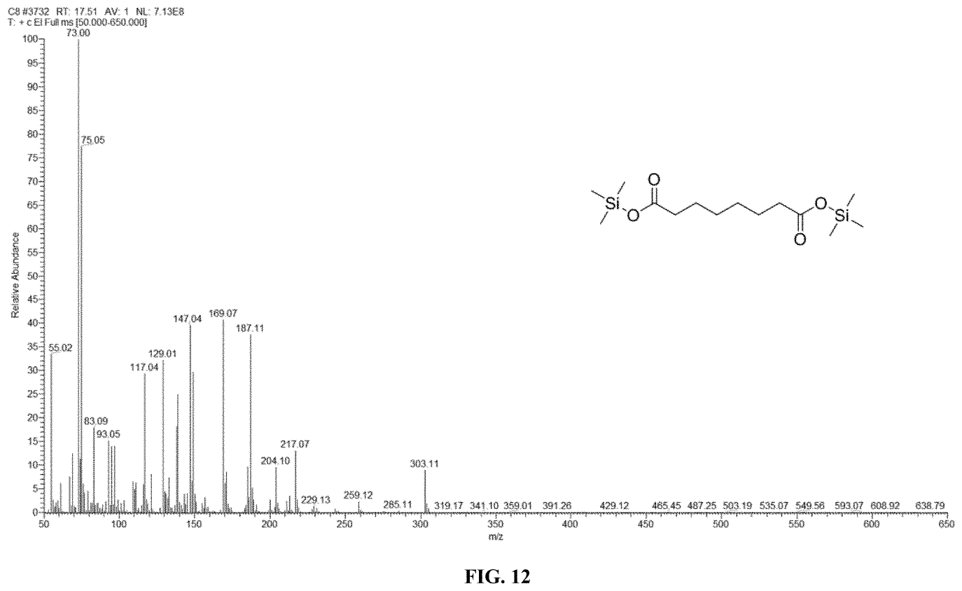

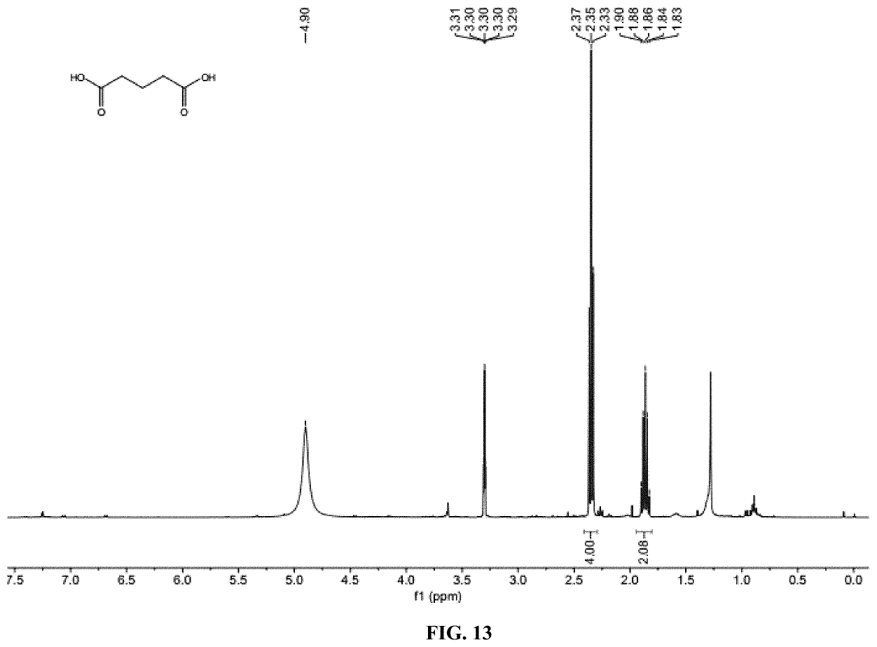

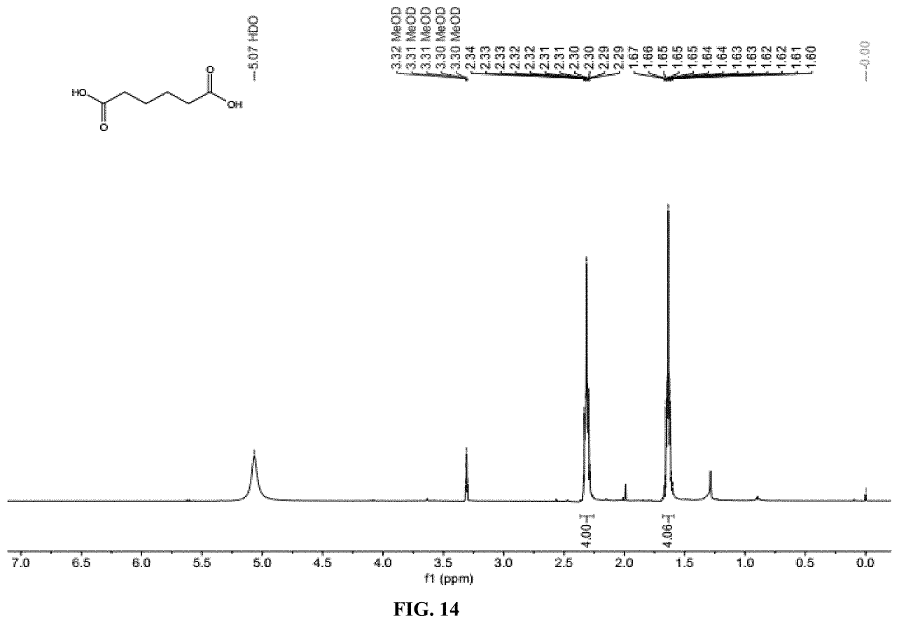

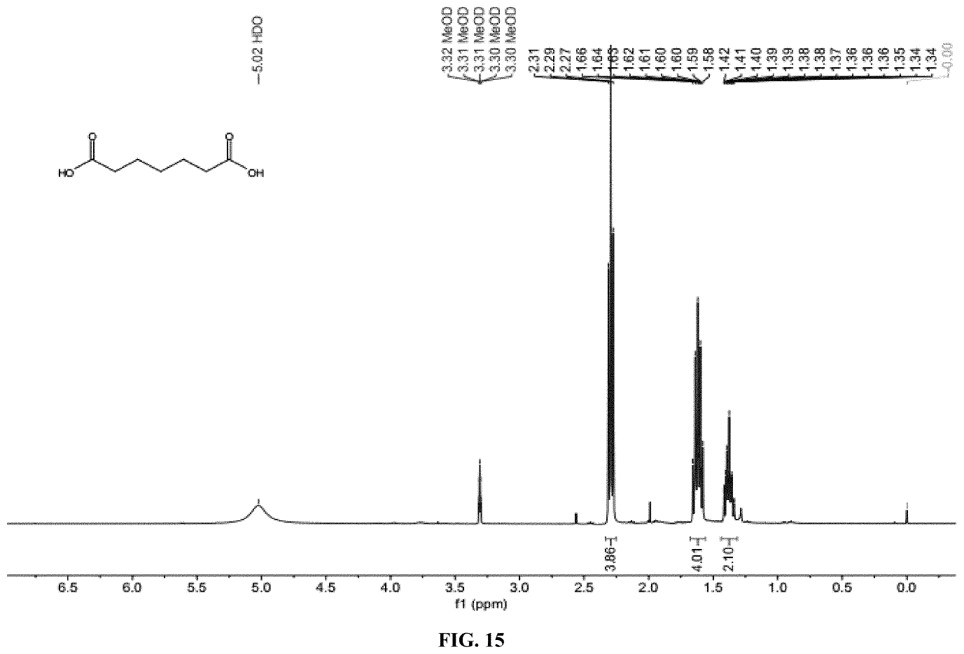

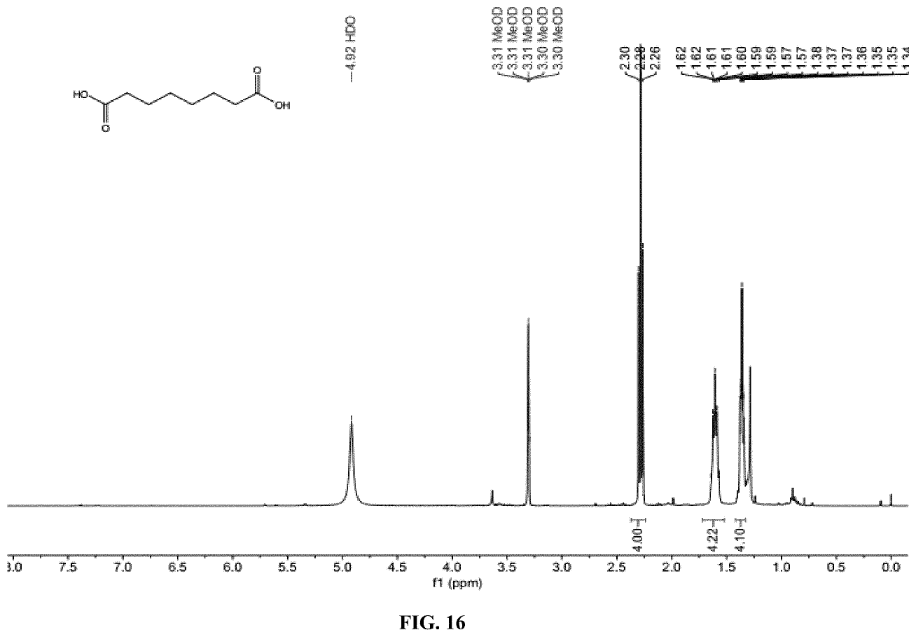

Four cycloalkanes (78 μL cyclopentane, 87 μL cyclohexane, 99 μL cycloheptane, or 109.8 μL cyclooctane) at a final concentration of 100 mM were added to 8 mL of E. coli MBS2 suspension (final OD₆₀₀ was 30, and the ratio of recombinant cells used for single expression module I, module II and module III was 2:1:2) and placed in a 250 mL shake flask at 25° C. and 200 rpm for reaction. 0.05 g/ml glucose was added for NADPH regeneration. After 24 hours of reaction, 2 mL of E. coli suspension used to express module III (resuspended in 200 mM pH 8.0 potassium phosphate buffer to OD₆₀₀ of 80) in order to ensure that 8-hydroxyoctanol could be completely converted into suberic acid. 10 M of NaOH was used to adjust the pH of the reaction system during the reaction to maintain it at about 8.0. After the reaction, the reaction mixture was extracted three times with 30 mL of ethyl acetate, and the substrates were distilled under reduced pressure for recovery. After that, 2 mL of 4 M HCL was added to adjust the aqueous phase to be about pH 1-2, 50 mL ethyl acetate was used to extract three times, the organic phase was collected and dried with anhydrous Na₂SO₄, and the solvent was distilled by rotary evaporator. The white solid was obtained as the corresponding α, ω-acid, with a purity of more than 98%, and finally, glutaric acid obtained was 13.4 mg, yield was 13%; adipic acid: 38.5 mg, yield was 33%; pimelic acid: 57.8 mg, yield was 45%; suberic acid: 18.8 mg, yield was 13%. Then the separated products were analyzed by GC-MS and NMR. The experimental spectrum is shown in FIG. 9-FIG. 16; Wherein, FIG. 9-FIG. 12 are the GC-MS spectrum of the silanization products after derivatization with glutaric acid, adipic acid, pimelic acid and suberic acid; FIG. 13 is ¹H NMR spectrum of glutaric acid, 7a: 1H NMR (400 MHz, CD3OD): δ 2.35 (t, J=7.4 Hz, 4H), 1.86 (p, J=7.4 Hz, 2H). FIG. 14 is ¹H NMR spectrum of adipic acid, 7b: ¹H NMR (400 MHz, CD3OD): δ 2.31 (ddt, J=7.5, 5.7, 2.1 Hz, 4H), 1.68-1.59 (m, 4H). FIG. 15 is ¹H NMR spectrum of pimelic acid, 7c: ¹H NMR (400 MHz, CD3OD): δ 2.29 (t, J=7.4 Hz, 4H), 1.62 (p, J=7.5 Hz, 4H), 1.44-1.31 (m, 2H). FIG. 16 is ¹H NMR spectrum of suberic acid, 7d: 1H NMR (400 MHz, CD3OD): δ 2.28 (t, J=7.4 Hz, 4H), 1.72-1.52 (m, 4H), 1.36 (m, 4H).

Wherein, yield=weight of the actual product/weight of the product theoretically obtained after the substrate is completely converted×100%

Test Methods for Products:

Derivatization treatment of samples: The organic phase samples obtained in Examples 4-9 were centrifuged at 13860×g for 10 min to remove the anhydrous Na₂SO₄ used for drying, and 300 μL of the samples were placed in a fume hood under normal temperature and pressure to volatilize ethyl acetate, and the white solid appearing at the bottom of the tube after volatilization was dissolved in 60 μL of pyridine and 30 μL of derivatization reagent N-methyl-N-(trimethylsilyl) trifluoroacetamide (MSTFA) was added. The derivatization reaction was carried out at 65° C. for 1 h, and then the mixture was used for GC analysis.

GC analysis conditions: In order to detect hydroxy acids and DCAs, derivatized samples were GC analyzed using SH-Rtx-1 column: 90 μL of ethyl acetate containing an internal standard (25 mM n-decane) was added to the derivative mixture. After that, the samples were analyzed using SHIMADZU Nexis GC-2030 system equipped with FID detector and SH-Rtx-1 column (30 m×0.25 mm, 0.25 μm). The temperature of the injector and detector were 250° C. and 280° C., respectively. The temperature program was as follows: from 50° C. to 120° C. at 5° C./min, raised to 240° C. at 40° C./min, and kept at 240° C. for 1 min.

In order to detect cycloalkanes, cycloalkanols, cycloalkanones and lactones, the obtained samples were analyzed by GC using a SH-Rtx-WAX column: the samples were analyzed using SHIMADZU Nexis GC-2030 system equipped with FID detector and SH-Rtx-1 column (30 m×0.25 mm, 0.25 μm). The temperature of the injector and detector were 250° C. and 280° C., respectively. The temperature program was as follows: from 50° C. to 120° C. at 5° C./min, raised to 240° C. at 40° C./min, and kept at 240° C. for 3 min.

The above described are only preferred embodiments of the present disclosure and are not intended to limit the present disclosure, any modifications, equivalent replacement and improvements made within the spirit and principle of the present disclosure should be regarded as the protection scope of the present disclosure.

1. A method for producing α, ω-dicarboxylic acids with whole-cell biocatalysis, comprises the steps of:
under normal temperature and pressure and aerobic conditions, catalytically converting substrate lactones to obtain α, ω-dicarboxylic acids by recombinant cells containing functional genes related to the pathway for catalyzing lactones to produce α, ω-dicarboxylic acids.

2. The method for producing α, ω-dicarboxylic acids according to claim 1, wherein the method further comprises recombinant cells containing functional genes related to the pathway for catalyzing cycloalkanol to produce lactones when cycloalkanols are used as substrates;
the functional genes related to the pathway for catalyzing cycloalkanol to produce lactones and the functional genes related to the pathway for catalyzing lactones to produce α, ω-dicarboxylic acids are located in the same cell, and the catalysis and transformation of substrates are realized by using a single cell system;
or the functional genes related to the pathway for catalyzing cycloalkanol to produce lactones and the functional genes related to the pathway for catalyzing lactones to produce α, ω-dicarboxylic acids are respectively constructed in different cells, and the catalysis and transformation of substrates are realized by using a multi-cell combination system.

3. The method for producing α, ω-dicarboxylic acids according to claim 2, wherein the method further comprises recombinant cells containing functional genes related to the pathway for catalyzing cycloalkane to produce cycloalkanol when cycloalkanes are used as substrates;
the functional genes related to the pathway for catalyzing cycloalkane to produce cycloalkanol, the functional genes related to the pathway for catalyzing cycloalkanol to produce lactones and the functional genes related to the pathway for catalyzing lactones to produce α, ω-dicarboxylic acids are located in the same cell, and the catalysis and transformation of substrates are realized by using a single cell system;
or the functional genes related to the pathway for catalyzing cycloalkane to produce cycloalkanol, the functional genes related to the pathway for catalyzing cycloalkanol to produce lactones and the functional genes related to the pathway for catalyzing lactones to produce α, ω-dicarboxylic acids are respectively located in different cells, and the catalysis and transformation of substrates are realized by using a multi-cell combination system;
or the functional genes related to the pathway for catalyzing cycloalkane to produce cycloalkanol, the functional genes related to the pathway for catalyzing cycloalkanol to produce lactones and the functional genes related to the pathway for catalyzing lactones to produce α, ω-dicarboxylic acids, any two of which are located in the same cell, and the other one is located in an another cell, the catalysis and transformation of substrates are realized by using a multi-cell combination system.

4. The method for producing α, ω-dicarboxylic acids according to claim 1, wherein the cells are selected from prokaryotic cells Escherichia coli, Corynebacterium glutamicum, Bacillus subtilis, Brevibacterium flavum, Serratia marcescens, and lower Eukaryotic Cells Saccharomyces cerevisiae.

5. The method for producing α, ω-dicarboxylic acids according to claim 4, wherein the α, ω-dicarboxylic acids comprise different dicarboxylic acids of a C5, C6, C7, C8, C10, C12 and C15.

6. The method for producing α, ω-dicarboxylic acids according to claim 5, wherein the functional genes related to the pathway for catalyzing lactones to produce α, ω-dicarboxylic acids comprise a lactonase gene, an alcohol dehydrogenase gene, an aldehyde dehydrogenase gene and a NADH oxidase gene.

7. The method for producing α, ω-dicarboxylic acids according to claim 2, wherein the functional genes related to the pathway for catalyzing cycloalkanol to produce lactones comprise an alcohol dehydrogenase gene and a Baeyer-Villiger monooxygenase gene.

8. The method for producing α, ω-dicarboxylic acids according to claim 3, wherein the functional genes related to the pathway for catalyzing cycloalkane to produce cycloalkanol are selected from a P450BM319A12 gene, a P450BM3 A82F gene and a P450BM3 A82F/A328F gene, further comprise a glucose dehydrogenase gene GDH.

9. The method for producing α, ω-dicarboxylic acids according to claim 5, comprises the following steps of: culturing the cells containing functional genes related to the pathway for catalyzing corresponding substrates to produce α, ω-dicarboxylic acids in TB liquid medium, and adding an inducer to induce expression; collecting the cultured cells and adding to a catalytic reaction system containing substrates for catalysis and conversion; adding glucose solution into the catalytic reaction system when the substrates are cycloalkane.

10. The method for producing α, ω-dicarboxylic acids according to claim 9, wherein the catalytic reaction is carried out under normal temperature, normal pressure and aerobic conditions, the catalytic reaction is carried out in a temperature range of 20° C. to 40° C.; preferably in a temperature range of 25° C.-30° C.; and more preferably at 25° C.

11. A method for producing α, ω-dicarboxylic acids, wherein using the whole-cell biocatalysis method for producing α, ω-dicarboxylic acids in claim 10.

12. The method for producing α, ω-dicarboxylic acids according to claim 11, wherein the α, ω-dicarboxylic acids comprise C5, C6, C7 or C8 diacid products.

13. A method for producing immobilized cells, wherein using the recombinant cells in the method for producing α, ω-dicarboxylic acids in claim 1.

14. The whole-cell biocatalysis method for producing α, ω-dicarboxylic acids according to claim 2, wherein the cells are selected from prokaryotic cells Escherichia coli, Corynebacterium glutamicum, Bacillus subtilis, Brevibacterium flavum, Serratia marcescens, and lower Eukaryotic Cells Saccharomyces cerevisiae.

15. The whole-cell biocatalysis method for producing α, ω-dicarboxylic acids according to claim 3, wherein the cells are selected from prokaryotic cells Escherichia coli, Corynebacterium glutamicum, Bacillus subtilis, Brevibacterium flavum, Serratia marcescens, and lower Eukaryotic Cells Saccharomyces cerevisiae.

16. The whole-cell biocatalysis method for producing α, ω-dicarboxylic acids according to claim 6, comprises the following steps of: culturing the cells containing functional genes related to the pathway for catalyzing corresponding substrates to produce α, ω-dicarboxylic acids in TB liquid medium, and adding an inducer to induce expression; collecting the cultured cells and adding to a catalytic reaction system containing substrates for catalysis and conversion; adding glucose solution into the catalytic reaction system when the substrates are cycloalkane.

17. The whole-cell biocatalysis method for producing α, ω-dicarboxylic acids according to claim 7, comprises the following steps of: culturing the cells containing functional genes related to the pathway for catalyzing corresponding substrates to produce α, ω-dicarboxylic acids in TB liquid medium, and adding an inducer to induce expression; collecting the cultured cells and adding to a catalytic reaction system containing substrates for catalysis and conversion; adding glucose solution into the catalytic reaction system when the substrates are cycloalkane.

18. The whole-cell biocatalysis method for producing α, ω-dicarboxylic acids according to claim 8, comprises the following steps of: culturing the cells containing functional genes related to the pathway for catalyzing corresponding substrates to produce α, ω-dicarboxylic acids in TB liquid medium, and adding an inducer to induce expression; collecting the cultured cells and adding to a catalytic reaction system containing substrates for catalysis and conversion; adding glucose solution into the catalytic reaction system when the substrates are cycloalkane.


SEQUENCE LISTING (33 sequences)

SEQ ID NO: 1 - DNA, 22 bp, Artificial Sequence
  pRSFDuet-1-F
gccaggatccgaattcgagctc

SEQ ID NO: 2 - DNA, 25 bp, Artificial Sequence
  pRSFDuet-1-R
gtggtgatgatggtgatggctgctg

SEQ ID NO: 3 - DNA, 45 bp, Artificial Sequence
  19A12_homologous seq-Fwd
caccatcatcaccacgcaattaaagaaatgcctcagccaaaaacg

SEQ ID NO: 4 - DNA, 44 bp, Artificial Sequence
  19A12_RBS-Rev
gatatatctccttaggtaccttacccagcccacacgtcttttgc

SEQ ID NO: 5 - DNA, 67 bp, Artificial Sequence
  RBS_GDH-Fwd
ggtacctaaggagatatatcatgtatacagatttaaaagataaagtagtagtaattacaggtggatc

SEQ ID NO: 6 - DNA, 43 bp, Artificial Sequence
  RBS_GDH-R
gctcgaattcggatcctggcttatccgcgtcctgcttggaatg

SEQ ID NO: 7 - DNA, 43 bp, Artificial Sequence
  ADH1_homologous seq-Fwd
caccatcatcaccacatgagcaatcgtctggatggtaaagttg

SEQ ID NO: 8 - DNA, 47 bp, Artificial Sequence
  ADH1_RBS-Rev
gatatatctccttaggtaccttactgtgcggtataaccaccatccac

SEQ ID NO: 9 - DNA, 53 bp, Artificial Sequence
  RBS_BVMO-Fwd
ggtacctaaggagatatatcatgtcacaaaaaatggattttgatgctatcgtg

SEQ ID NO: 10 - DNA, 43 bp, Artificial Sequence
  BVMO_homologous seq-Rev
gaattcggatcctggcttaggcattggcaggttgcttgatatc

SEQ ID NO: 11 - DNA, 46 bp, Artificial Sequence
  ADH2_homologous seq-Fwd
gccatcaccatcatcaccaccattgttattgcgttacccatcatgg

SEQ ID NO: 12 - DNA, 47 bp, Artificial Sequence
  ADH2_RBS-Rev
gatatatctccttaggtaccttagttctcgtgcatcagaacgatacg

SEQ ID NO: 13 - DNA, 54 bp, Artificial Sequence
  RBS_ ALDH-Fwd
ggtacctaaggagatatatcatgaactatccgaatattccgctgtatattaacg

SEQ ID NO: 14 - DNA, 48 bp, Artificial Sequence
  ALDH_homologous seq-Rev
gctcgaattcggatcctggcttagttcagctgggtgataaatttggtg

SEQ ID NO: 15 - DNA, 46 bp, Artificial Sequence
  Lactonase_homologous seq-Fwd
gccatcaccatcatcaccacaccaatattagcgaaaccctgagcac

SEQ ID NO: 16 - DNA, 49 bp, Artificial Sequence
  Lactonase_RBS-Rev
gatatatctccttaggtaccttattccagggctttctgataccatgctg

SEQ ID NO: 17 - DNA, 54 bp, Artificial Sequence
  RBS_ NOX-Fwd
ggtacctaaggagatatatcatgaaagttatcgtaattggttgtactcatgccg

SEQ ID NO: 18 - DNA, 49 bp, Artificial Sequence
  NOX_homologous seq-Rev
gctcgaattcggatcctggcttattccgtcactttttcagccgcatgag

SEQ ID NO: 19 - DNA, 22 bp, Artificial Sequence
  M3B-F
gccaggatccgaattcgagctc

SEQ ID NO: 20 - DNA, 48 bp, Artificial Sequence
  ALDH_RBS-Rev
gatatatctccttaggtaccttagttcagctgggtgataaatttggtg

SEQ ID NO: 21 - DNA, 47 bp, Artificial Sequence
  RBS_ Lactonase-Fwd
ggtacctaaggagatatatcatgaccaatattagcgaaaccctgagc

SEQ ID NO: 22 - DNA, 49 bp, Artificial Sequence; identical to SEQ ID NO: 18
  NOX_ homologous seq-Rev

SEQ ID NO: 23 - DNA, 22 bp, Artificial Sequence
  M1D-F
gccaggatccgaattcgagctc

SEQ ID NO: 24 - DNA, 43 bp, Artificial Sequence
  GDH_RBS-Rev
gatatatctccttaggtaccttatccgcgtcctgcttggaatg

SEQ ID NO: 25 - DNA, 48 bp, Artificial Sequence
  RBS_ ADH1-Fwd
ggtacctaaggagatatatcatgagcaatcgtctggatggtaaagttg

SEQ ID NO: 26 - DNA, 43 bp, Artificial Sequence; identical to SEQ ID NO: 10
  BVMO_ homologous seq-Rev

SEQ ID NO: 27 - DNA, 786 bp, Artificial Sequence
  Gene Sequence(GDH)
atgtatacagatttaaaagataaagtagtagtaattacaggtggatcaacaggtttaggacgcgcaatggctgttcgtttcggtcaagaagaagcaaaagttgttattaactattacaacaatgaagaagaagctttagatgcgaaaaaagaagtagaagaagcaggcggacaagcaatcatcgttcaaggcgacgtaacaaaagaagaagatgttgtaaaccttgttcaaacagctattaaagaattcggtacattagacgttatgattaataacgctggtgttgaaaacccagttccttctcatgagttatctttagacaactggaataaagttattgatacaaacttaacaggtgcattcttaggaagccgtgaagcaatcaaatattttgttgaaaacgacattaaaggaaacgttattaacatgtctagtgttcatgaaatgattccttggccattatttgttcattacgcagcaagtaaaggcggtatgaaactaatgacggaaacattggctcttgaatatgcgccaaaaggtatccgcgtaaataacattggaccaggtgcgatgaacacaccaattaacgcagagaaatttgcagatcctgtacaacgtgcagacgtagaaagcatgattccaatgggttacatcggtaaaccagaagaagtagcagcagttgcagcattcttagcatcatcacaagcaagctatgtaacaggtattacattatttgctgatggtggtatgacgaaatacccatcattccaagcaggacgcggataa

SEQ ID NO: 28 - DNA, 759 bp, Artificial Sequence
  Gene Sequence(ADH1)
atgagcaatcgtctggatggtaaagttgcaattattaccggtggcaccttaggtattggtctggcaattgcaaccaaatttgttgaagagggtgccaaagttatgattaccggtcgtcatagtgatgttggtgaaaaagcagcaaaaagcgttggtacaccggatcagattcagttttttcagcatgatagcagtgatgaagatggttggaccaaactgtttgatgcaaccgaaaaagcatttggtccggttagcaccctggttaataatgcaggtattgcagtgaataagagcgttgaagaaaccaccaccgcagaatggcgtaaactgctggcagttaatctggatggcgttttttttggtacacgtctgggtattcagcgcatgaaaaacaaaggtctgggtgcaagcattatcaacatgagcagcattgaaggttttgttggtgatccgagcctgggtgcatataatgcaagcaaaggtgcagttcgtattatgagcaaaagcgcagcactggattgtgcactgaaagattatgatgttcgtgtgaataccgttcatccgggttatatcaaaacaccgctggttgatgatctgcctggtgccgaagaagcaatgagccagcgtacaaaaaccccgatgggtcatattggtgaaccgaatgatattgcctatatctgtgtttatctggccagcaacgaaagtaaatttgcaaccggtagcgaatttgttgtggatggtggttataccgcacagtaa

SEQ ID NO: 29 - DNA, 1632 bp, Artificial Sequence
  Gene Sequence(BVMO)
atgtcacaaaaaatggattttgatgctatcgtgattggtggtggttttggcggactttatgcagtcaaaaaattaagagacgagctcgaacttaaggttcaggcttttgataaagccacggatgtcgcaggtacttggtactggaaccgttacccaggtgcattgacggatacagaaacccacctctactgctattcttgggataaagaattactacaatcgctagaaatcaagaaaaaatatgtgcaaggccctgatgtacgcaagtatttacagcaagtggctgaaaagcatgatttaaagaagagctatcaattcaataccgcggttcaatcggctcattacaacgaagcagatgccttgtgggaagtcaccactgaatatggtgataagtacacggcgcgtttcctcatcactgctttaggcttattgtctgcgcctaacttgccaaacatcaaaggcattaatcagtttaaaggtgagctgcatcataccagccgctggccagatgacgtaagttttgaaggtaaacgtgtcggcgtgattggtacgggttccaccggtgttcaggttattacggctgtggcacctctggctaaacacctcactgtcttccagcgttctgcacaatacagcgttccaattggcaatgatccactgtctgaagaagatgttaaaaagatcaaagacaattatgacaaaatttgggatggtgtatggaattcagcccttgcctttggcctgaatgaaagcacagtgccagcaatgagcgtatcagctgaagaacgcaaggcagtttttgaaaaggcatggcaaacaggtggcggtttccgtttcatgtttgaaactttcggtgatattgccaccaatatggaagccaatatcgaagcgcaaaatttcattaagggtaaaattgctgaaatcgtcaaagatccagccattgcacagaagcttatgccacaggatttgtatgcaaaacgtccgttgtgtgacagtggttactacaacacctttaaccgtgacaatgtccgtttagaagatgtgaaagccaatccgattgttgaaattaccgaaaacggtgtgaaactcgaaaatggcgatttcgttgaattagacatgctgatactggccacaggttttgatgccgtcgatggcaactatgtgcgcatggacattcaaggtaaaaacggcttggccattaaagactactggaaagaaggtccgtcgagctatatgggtgtcaccgtaaataactatccaaacatgttcatggtgcttggaccgaatggcccgtttaccaacctgccgccatcaattgaatcacaggtggaatggatcagtgataccattcaatacacggttgaaaacaatgttgaatccattgaagcgacaaaagaagcggaagaacaatggactcaaacttgcgccaatattgcggaaatgaccttattccctaaagcgcaatcctggatttttggtgcgaatatcccgggcaagaaaaacacggtttacttctatctcggtggtttaaaagaatatcgcagtgcgctagccaactgcaaaaaccatgcctatgaaggttttgatattcaattacaacgttcagatatcaagcaacctgccaatgcctaa

SEQ ID NO: 30 - DNA, 1059 bp, Artificial Sequence
  Gene Sequence(ADH2)
atgcattgttattgcgttacccatcatggtcagccgctggaagatgttgaaaaagaaattccgcagccgaaaggcaccgaagttctgctgcatgttaaagcagcaggtctgtgtcataccgatctgcatctgtgggaaggttattatgatttaggtggtggtaaacgtctgagcctggcagatcgtggtctgaaaccgcctctgacactgagccatgaaattaccggtcaggttgttgcagttggtccggatgcagaaagcgttaaagttggtatggttagcctggttcatccgtggattggttgtggtgaatgtaattattgtaaacgcggtgaagaaaacctgtgtgcaaaaccgcagcagctgggtattgcaaaacctggtggttttgcagaatacattattgttccgcatccgcgttatctggttgatattgcaggtctggatctggccgaagcagcaccgctggcatgtgccggtgttaccacctatagcgcactgaaaaaattcggtgatctgattcagagcgaaccggttgttattattggtgccggtggtctgggtctgatggcactggaactgctgaaagcaatgcaggcaaaaggtgcaattgttgtggatatcgatgatagcaaactggaagcagcccgtgcagccggtgcactgagcgtgattaatagccgtagcgaagatgcagcacagcagctgattcaggccaccgatggtggtgcacgtctgattctggacctggttggtagcaatccgacactgagtctggcactggcaagcgcagcacgtggtggtcatattgttatttgtggcctgatgggtggtgaaatcaaactgagcattccggttattccgatgcgtccgctgaccattcagggtagctatgttggcaccgttgaagaactgcgtgaactggttgagctggttaaagaaacccatatgagcgcaattccggtgaaaaaactgccgattagccagattaatagtgcctttggcgatctgaaagatggtaatgttattggtcgtatcgttctgatgcacgagaactaa

SEQ ID NO: 31 - DNA, 1434 bp, Artificial Sequence
  Gene sequence(ALDH)
atgaactatccgaatattccgctgtatattaacggcgaatttctggatcataccaatcgtgatgtgaaagaagtgtttaacccggttaaccatgaatgcattggtctgatggcatgtgcaagccaggcagatctggattatgcactggaaagcagccagcaggcatttctgcgttggaaaaaaaccagtccgattacacgtagcgaaattctgcgtacctttgcaaaactggcacgtgaaaaagcagcagaaattggtcgcaatattaccctggatcagggcaaaccgctgaaagaagcaattgccgaagttaccgtttgtgcagaacatgcagaatggcatgcagaagaatgtcgtcgtatttatggtcgtgttattccgcctcgtaatccgaatgttcagcagctggttgttcgtgaaccgctgggtgtttgtctggcatttagcccgtggaattttccgtttaatcaggccattcgtaaaatcagcgcagcaattgcagcaggttgtaccattattgttaaaggtagcggtgataccccgagcgcagtttatgcaattgcccagctgtttcatgaagcaggtctgccgaatggtgttctgaatgttatttggggtgatagcaacttcatcagcgactatatgattaaaagcccgatcatccagaaaatcagctttaccggtagcacaccggttggtaaaaaactggccagccaggcaagcctgtatatgaaaccgtgtaccatggaattaggtggtcatgcaccggttattgtttgtgatgatgcagatattgatgcagccgttgaacatctggttggttacaaatttcgtaatgcaggtcaggtttgtgttagcccgacacgtttttatgttcaagagggcatctataaagagtttagcgaaaaagttgttctgcgtgccaagcagattaaagttggttgtggtctggatgcaagcagcgatatgggtccgctggcacaggcacgtcgtatgcatgcaatgcagcagatcgttgaagatgcagttcataaaggtagtaaactgctgttaggtggcaacaagattagcgataaaggcaacttttttgaaccgaccgttctgggtgatctgtgtaatgatacccagtttatgaacgatgaaccgtttggtccgattatcggtctgattccgtttgataccattgatcatgttctggaagaagcaaatcgtctgccgtttggcctggcaagctatgcatttaccaccagtagcaaaaatgcacaccagattagctatggtctggaagcaggtatggttagcattaaccatatgggtttagcactggcagaaaccccgtttggtggtattaaagatagtggttttggtagcgaaggtggcattgaaacctttgatggttatctgcgcaccaaatttatcacccagctgaactaa

SEQ ID NO: 32 - DNA, 942 bp, Artificial Sequence
  Gene sequence(Lactonase)
atgaccaatattagcgaaaccctgagcaccgcacctggtggtgcagcaggtccggatgttctgcgtgatctgtatgcagattggagcgaaattatggcagcaacaccggatctgaccattcgtctgctgcgtagcctgtttgatgaatggcatcagccgaccgttgaaccggaaggtgttacctatcgtgaagaaaccgttggtggtgttcctggtatttggtgtctgccgcagggtgcagatggtagcaaagttctgctgtatacccatggtggtggttttgcagttggtagcgcagcaagccatcgtaaactggcaggtcatgttgcaaaagcactgggtgccgttggttttgttctggattatcgtcgtgcaccggaatttcagcatccggcacagattgaagatggtgttgcagcatttgatgcactggttgcaaatggtattgcaccgcaggatattaccaccattggtgatagtgccggtggtaatctggcagttgcaattgccctgagcctgcgtgaacagggtaaacaaggtccgggtagcgttattgcatttagcccgtggctggatatggaaaataaaggtgaaaccctggccaccaataatgataccgatgcactgattacaccggaactgctggaaggcatgattgccggtgtgctgggtgataccattgatccgaaaacaccgctggcaaatccgctgtatgccgattttaccggttttccgcgtctgtatatcaccgcaggtagcgttgaaagcctgctggataatgcaacccgtctggaaaaattagcagcatctgccggtgttgatgttaccctgagtattggtgaaggtcagcagcatgtttatccgtttctggcaggccgtagcgcactggtggatgatgaatttgcaaagctggcagcatggtatcagaaagccctggaataa

SEQ ID NO: 33 - DNA, 1353 bp, Artificial Sequence
  Gene sequence(NOX)
atgaaagttatcgtaattggttgtactcatgccggaactgctgctgtaaatcaaatcttggcgtcaaatccagaaacagacgtcacgatttatgaacggaatgacaatgtgtcatttctctcctgtgggattgccctctatcttggtggcgaagttgccgatccacaagggctcttctattccagtccagaacaattagccaaattaggcgcgaatgttcatatgcaacatgatgtgaccgacgtggataccgaaaatcatgaaattaccgttactgatttgaagaccggcgaatccaagaaagattattacgacaaattagttgtcacaactggttcatggcctgtaattccaccaatcgatggtatcgacagcccgaacgtttacctctgcaagaactggacgcatgcccaaagtttatgggaagctgccaagccagctaagcgcgtcatcgttatcggtgggggctacattgggactgaattagtcgaagcttatcagaagcaaggtaaggaagttaccttaattgatggcttaccacggattttaaacaagtatttagacaaaggcttcactgaccgggtcgaaaaagacttcgttgaccatggcatcaagatggccttaaatcagatggttaaaggcttcagtgatgatggcaaggaagttaccgttaagactgacaagggcagctacaccgctgatatggcaattctctgtgttggtttccggccaaacaccagcctattaaagggcaaagttgacatgaacccgaacggctctattaagacaaatgactacatgcaaacatctgaccctgatatctacggtgctggtgattccgttgcggttcactacaacccaactaagaaggatgcctacattccattagccactaacgcggttcgccaagggactttagttggtttgaacatcttcaagccaacccggaagtacatggggacgcaatcaacttctggtttaatgttattcggcaagacgatcgtttcttctgggatgaccttggaacatgctcaagctgaaaaggtacctgcagaagccgttacctttgaagataactaccgtccagaatttatgccaaccacgaaaccagttctgatgcaattggtttacaacccagagacgcgtgaaatcttaggggcccaattcatgagtgaacatgacgtttcacaatcggctaacgtgatctcagtgatgattcaaaatcacaacacgatcgatgacttaggctttgttgacatgttcttccagccaatctatgaccgtccattcaactacttgaacttattaggccaagcagccatcgctcatgcggctgaaaaagtgacggaataa